(19) United States
(12) Patent Application Publication
(10) Pub. No.: US 2004/0143104 A1
(43) Pub. Date: Jul. 22, 2004

(54) Methods of treating diabetes and other blood sugar disorders

(76) Inventors: Samuel C. Wadsworth, Shrewsbury, MA (US); Donna Armentano, Belmont, MA (US); Richard J. Gregory, Westford, MA (US); Geoffrey Parsons, Jamaica Plain, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 10/716,326

(22) Filed: Nov. 17, 2003

Related U.S. Application Data
(63) Continuation-in-part of application No. 10/215,272, filed on Aug. 7, 2002.
(60) Provisional application No. 60/310,982, filed on Aug. 8, 2001.

(51) Int. Cl. C07K 14/605; C07H 21/04; C12P 21/04

(52) U.S. Cl. 530/399; 435/69.7; 435/320.1; 435/325; 530/351; 536/23.5

(57) ABSTRACT

Compositions, expression vectors and host cells comprising nucleic acid which encodes a precursor glucagon-like peptide 1 (GLP-1) comprising mammalian GLP-1 linked to a heterologous signal sequence are encompassed by the present invention. The invention also relates to a method of promoting insulin production in an individual comprising administering to the individual an effective amount of a nucleic acid encoding a precursor GLP-1. The present invention also relates to a method of treating an individual having a blood sugar defect (e.g., type I or type II diabetes), comprising administering to the individual an effective amount of a nucleic acid encoding the precursor GLP-1. In a particular embodiment, the invention pertains to a method of treating an individual having a blood sugar defect comprising administering to the individual an effective amount of a nucleic acid encoding a precursor GLP-1 wherein the precursor GLP-1 comprises a signal sequence which codes for precursor cleavage at the activation cleavage site of the precursor GLP-1.

### RELATED APPLICATION(S)

This application is a continuation in part of U.S. application Ser. No. 10/215,272 filed Aug. 7, 2002 which claims benefit of U.S. Provisional Application No. 60/310,982, filed Aug. 8, 2001. The entire teachings of the above application(s) are incorporated herein by reference.

### BACKGROUND OF THE INVENTION

In general terms, the most common types of diabetes mellitus are Type I, Impaired Glucose Tolerance (“IGT”) and Type II. In Type I diabetes, the beta cells in the pancreas, probably through an auto-immune reaction, cease producing insulin into the bloodstream of the person. Insulin is a chemical substance which is normally secreted into the bloodstream by beta cells within the pancreas. Insulin is vitally important to the person because it enables the person to properly utilize and consume sugar in the bloodstream as part of the metabolism process.

Two major forms of diabetes mellitus are now recognized. Type I diabetes, 15 or insulin-dependent diabetes, is the result of an absolute deficiency of insulin, the hormone which regulates glucose utilization. Type II diabetes, or non-insulin-independent diabetes, often occurs in the face of normal, or even elevated levels of insulin and appears to be the result of the inability of tissues to respond appropriately to insulin. Most of the Type II diabetics are also obese. In Type I cases, where the pancreas has ceased producing insulin, it is necessary for the afflicted person to inject insulin directly into the bloodstream at prescribed periodic intervals and dosages in order to control the level of sugar in the blood. This is called intravenous injection. Oral ingestion of insulin is also possible but usually less effective due to the degradation of insulin caused by the passage through the stomach and upper intestine.

In IGT and Type II diabetes, the pancreas continues to produce insulin but, some or all of the insulin may fail to bind to the body cell receptors and/or internalization of insulin in the cells is reduced. In such cases, there may be a sufficient level of insulin in the blood, but the ability of the cells to uptake glucose is reduced or non-existent because of reduced internalized insulin. In a Type II diabetic cells to which insulin is bound does not take up glucose indicating a defect in the signaling pathway. This results in an increased need for insulin; however, this need for insulin is not met because the P cells in a Type II diabetic are defective in that they do not secrete enough insulin.

The existence of Type I, IGT or Type TI diabetes in a person is usually determined by an oral glucose tolerance test (OGTT). OGTT is a test in which the fasting patient is given a known amount of glucose (sugar) by mouth, and the blood is tested at intervals thereafter to note the quantity of sugar in the blood. A curve is then constructed from which important information about the person can be drawn. The glucose tolerance test curve will typically show whether the patient is hyperglycaemic (diabetic) or whether the patient has too little sugar in his or her blood and is therefore hypoglycaemic.

Symptoms of hyperglycaemia can be headaches, increased urination, thirst, nausea, weight loss, fatigue and coma. Hyperglycaemia can be caused by Hypoinsulinism, a condition in which the insulin producing beta cells of the pancreas fail to manufacture insulin or manufacture arid secrete a reduced amount of insulin into the bloodstream. In such cases, levels of sugar in the blood are dramatically increased.

Hyperglycaemia can also be caused by failure of some or all of the available insulin 25 in the blood to bind to the body's cell receptors and/or internalization of insulin in the cells is reduced. Hypoglycaemia (too little sugar) is also a blood condition that diabetics must constantly guard against. The symptoms of hypoglycaemia are abrupt episodes of intense hunger, trembling of the hands and body, faintness, black spots before the eyes, mental confusion, sweating, abnormal behavior, and, in severe cases, convulsions with loss of consciousness. In such cases, examination of the blood at the time of these attacks will show an extremely low level of circulating sugar in the blood.

Insulin dependent diabetes mellitus (IDDM) is an organ specific autoimmune disease affecting close to a million people in different age groups in the United States. The disease is characterized by extensive destruction of the insulin producing beta cells in the pancreatic islets and dysregulation of glucose metabolism leading to frank diabetes. The defining feature of IDDM is the lymphocytic infiltration of the islets. Among the invading cells, T cells appear to be one of the major mediators of autoimmune destruction. Type I diabetes is further characterized by increased levels of antibodies to various islet associated antigens, including insulin, GAD65, GAD67 and ICA5 12. These antibodies can be detected much before frank disease, and an immune response to such antigens can be used as a predictor for impending diabetes in patients with susceptible genetic (HLA) haplotypes. Currently, patients are dependent on insulin injections to maintain normoglycemia.

Insulin is a polypeptide hormone consisting of two disulfide-linked chains, an A chain consisting of 21 amino acid residues and a B chain of 30 residues. While administration of insulin can provide significant benefits to patients suffering from diabetes, the short serum half-life of insulin creates difficulties for maintaining proper dosage. The use of insulin also can result in a variety of hypoglycemic side-effects and the generation of neutralizing antibodies. Lee et al., Nature 408:483-488 (2000) have created a single-chain insulin analog (SIA), which does not need to be processed, and thus is relatively simple to make recombinantly. Others, such as Thule et al. Gene Therapy 7:1744-1752 (2000) have engineered an insulin chain that is processed by furin, a ubiquitously expressed endoprotease.

Type II diabetes is a progressive, multifactorial disease which results from insulin resistance and is characterized initially by elevated fasting blood glucose levels. It is believed that genetic factors contribute to susceptibility to type II diabetes, but other important risk factors such as, obesity, aging, diet, and lack of exercise also play a role. A large number of drugs have been developed to treat hyperglycemia, including those that promote release of insulin from the pancreas, uptake of glucose from the blood, and reduction in the level of glucose production. Unfortunately, these treatments generally only slow the progression of type II diabetes, which can progress to an insulin dependent state and the development of complications associated with diabetes such as hypertension, problematic ulcerative lesions on limbs, end-stage renal failure, retinopathy and cardiovascular disease. New therapies, especially therapies that can halt disease progression and, thus, its complications are urgently needed. More than 10 million people in the US alone suffer from type II diabetes, with the incidence increasing dramatically.

Proglucagon is expressed in a cells of the pancreas and in intestinal L cells but is proteolytically processed in these cell types to different peptide hormones that have opposing biological actions on glucose homeostasis. In a cells in the pancreas, proglucagon is processed to glucagon, which opposes the action of insulin, and in the intestinal endocrine L cells it is processed to glucagon-like peptide (GLP-1). This differential processing is due to differential expression of specific endoproteases belonging to the family of subtilisin-like pro-protein convertases. PC2 is expressed in the α cells in the pancreas whereas PC3 (also known as PC1) is expressed in the intestinal L cells.

Glucagon-like peptide (GLP-1) is released from the intestine in response to food uptake and has many activities. In its native form, GLP-1 is a 37 amino acid peptide known to inhibit neurons in the nervous system responsible for food and water intake. Tang-Christensen et al., Diabetes 47:530-537 (1998). In addition, GLP-1 is an insulinotropic molecule, however, only when an individual is in a hyperglycemic state.

Glucagon-like peptide 1 (GLP-1) is known to play a critical role in the regulation of the physiological response to feeding. GLP-1 is processed from proglucagon and is released into the blood from the endocrine L-cells mainly located in the distal small intestine and colon in response to ingestion of a meal. GLP-1 acts through a G protein-coupled cell surface receptor (GLP-1R) and enhances nutrient-induced insulin synthesis and release. GLP-1 stimulates insulin secretion (insulinotropic action) and cAMP formation. GLP-1(7-36) amide stimulates insulin release, lowers glucagon secretion, and inhibits gastric secretion and emptying. These gastrointestinal effects of GLP-1 are not found in vagotomized subjects, pointing to a centrally-mediated effect. GLP-1 binds with high affinity, to isolated rat adipocytes, activating cAMP production and stimulating lipogenesis or lipolysis. GLP-1 stimulates glycogen synthesis, glucose oxidation, and lactate formation in rat skeletal muscle.

Other important properties of GLP-1 include its ability to promote (cell differentiation and replication, thus aiding in the preservation of pancreatic islets, and its ability to inhibit gluconeogenesis in the liver.

Messenger RNA encoding the pancreatic-type GLP-1 receptor is found in relatively high quantities in rat pancreatic islets, lung, hypothalamus, and stomach. Interestingly, despite the knowledge that both GLP-1 and GLP-1 receptors are found in the hypothalamus, no central role for GLP-1 was determined until a recent report that GLP-1 administered by the intracerebroventricular route (ICV) markedly inhibits feeding in fasted rats. The same report indicates that after ICV administration of GLP-1, c-fos, a marker of neuronal activation, appears exclusively in the paraventricular nucleus of the hypothalamus and in the central nucleus of the amygdala, two regions of the brain of primary importance in the regulation of feeding. ICV GLP-1 also significantly reduces food intake following injection of the powerful feeding stimulant, neuropeptide Y, in animals fed ad libitum. A subsequent report demonstrates that GLP-1 administered centrally or peripherally is involved in control of body temperature regulation, but does not affect food intake after acute intraperitoneal administration in rats. A recent article reports that lateral ventricular injections of GLP-1 in sated rats induce extensive stimulation of Fos-ir in the paraventricular nucleus and parvocellular central nucleus of the amygdala, substantiating Turton, et al. Additionally, these investigators described strong activation of other centers involved in the regulation of feeding, including the immediate early gene protein product in the nucleus of the tractus solitarius, the pontine lateral parabrachial nucleus, the basal nucleus of the stria terminals, and the area postrema. GLP-1 receptors accessible to peripheral GLP-1 are found in the rat subfornical organ and area postrema.

Turton et al. (1996) specifically state that the effects of GLP-1 on body weight and food intake are caused only by administration of GLP-1 directly in the cerebroventriculum, that intraperitoneal administration of GLP-1, even at relatively high does, does not affect early dark-phase feeding, and that GLP-1 fragments are inactive when administered peripherally. Such statements discourage the use of GLP-1 as a composition (pharmaceutical agent) for reducing body weight, because central routes of administration, such as the ICV route, are not feasible for treating obesity in humans. The physiological effects of GLP-1 documented above have led to the suggestion of its beneficial use for treating diabetes and obesity by transplanting recombinant cell lines encoding GLP-1 or GLP or GLP-1 receptors, for example (WO 96/25487).

However, an urgent need exists for alternative, effective therapies for blood disorders such as diabetes.

### SUMMARY OF THE INVENTION

The present invention relates to compositions which can be used to treat blood sugar disorders such as diabetes. More particularly, the present invention relates to nucleic acids which encode a glucagon-like 1 peptide (GLP-1), vectors comprising the nucleic acids and methods in which the compositions are administered to an individual to promote (stimulate) insulin production. Thus, the compositions of the present invention provide for the treatment of blood sugar disorders.

In particular, the present invention relates to an isolated nucleic acid which encodes a precursor glucagon-like peptide 1 (GLP-1) comprising mammalian GLP-1 linked to a heterologous signal sequence.

In one embodiment, the mammalian GLP-1 encoded by the isolated nucleic acid has the amino acid sequence of GLP-1 7-37 (SEQ ID NO: 21). In another embodiment, the GLP-1 is a modified GLP-1. For example, the modified GLP-1 encoded by the nucleic acid has an amino acid sequence in which alanine at position 8 of GLP-1 7-37 is replaced with glycine (SEQ ID NO: 22). Other examples of modified GLP-1 include GLP-1 having an amino acid sequence selected from the group consisting of: GLP-1 (7-34) (SEQ ID NO: 23), GLP-1 (7-35) (SEQ ID NO: 24), GLP-1(7-36) (SEQ ID NO: 25), Val⁸-GLP-1(7-37) (SEQ ID NO: 26), Gln⁹-GLP-1(7-37) (SEQ ID NO: 27), Thr¹⁶-Lys¹⁸-GLP-1(7-37) (SEQ ID NO: 28), Lys¹⁸GLP-1(7-37) (SEQ ID NO: 29) and D-Gln⁹ (7-37) (SEQ ID NO: 30).

The heterologous signal sequence which is linked to the mammalian GLP-1 comprises a cleavage site which is cleaved by a peptide (e.g., a protease). For example, the heterologous signal sequence can be a (one or more) signal peptide sequence and/or a leader sequence. The heterologous signal sequence can be derived from a protein such as a cytokine, a growth factor, a colony stimulating factor, a clotting factor, a protein of a (PACAP)/Glucagon superfamily and a serum protein. For example, the heterologous signal sequence can be derived from a secreted human alkaline phosphatase (SEAP) signal peptide sequence, a proexendin 4 leader sequence, a pro-helodermin leader sequence, a pro-glucose dependent insulinotropic polypeptide (GIP) leader sequence, a pro-insulin-like growth factor 1 (IGFI) leader sequence, a preproglucagon leader sequence, an alpha-1 antitrypsin leader sequence, a human lymphotoxin A leader sequence, or a human clusterin leader sequence. In a particular embodiment, the heterologous signal sequence comprises a furin cleavage site. The furin cleavage site can encode a peptide such as Arg-X-Lys-Arg (SEQ ID NO: 34), Arg-X-Arg-Arg (SEQ ID NO: 35), Lys/Arg-Arg-X-Lys/Arg-Arg (SEQ ID NO: 36) and Arg-X-X-Arg (SEQ ID NO: 37) such as an Arg-Gln-Lys-Arg (SEQ ID NO: 38). The heterologous signal sequence can also comprise a prohormone convertase (PC) cleavage site.

In one embodiment, the isolated nucleic acid which encodes a precursor GLP-1 comprising mammalian GLP-1 linked to a heterologous signal sequence is selected from the group consisting of SEQ ID NO: I; SEQ ID NO: 3; SEQ ID NO: 5; SEQ ID NO: 7; SEQ ID NO: 9; SEQ ID NO: 11; SEQ ID NO: 13; SEQ ID NO: 15; SEQ ID NO: 17; and SEQ ID NO: 19.

In another embodiment, the isolated nucleic acid which encodes a precursor GLP-1 comprising mammalian GLP-1 linked to a heterologous signal sequence, encodes an amino acid sequence selected from the group consisting of: SEQ ID NO: 2; SEQ ID NO: 4; SEQ ID NO: 6; SEQ ID NO: 8; SEQ ID NO: 10; SEQ ID NO: 12; SEQ ID NO: 14; SEQ ID NO: 16; SEQ ID NO: 18; and SEQ ID NO: 20.

The present invention also relates to an isolated precursor GLP-1 comprising mammalian GLP-1 linked to a heterologous signal sequence. In one embodiment, the present invention also relates to an isolated polypeptide encoded by a nucleic acid described herein. The precursor GLP-1 can comprise a GLP-1 (e.g., SEQ ID NO: 21) or a modified GLP-1 (e.g., modified GLP-1 having an amino acid sequence in which alanine at position 8 is replaced with glycine (SEQ ID NO: 22), GLP-1 (7-34) (SEQ ID NO: 23), GLP-1(7-35) (SEQ ID NO: 24), GLP-1(7-36) (SEQ ID NO: 25), Val⁸-GLP-1(7-37) (SEQ ID NO: 26), Gln⁹-GLP-1(7-37) (SEQ ID NO: 27), Thr¹⁶-Lys¹⁸-GLP-1 (7-37) (SEQ ID NO: 28), Lys¹⁸ (SEQ ID NO: 29) and D-Gln⁹GLP-1 (7-37) (SEQ ID NO: 30).

The heterologous signal sequence of the precursor GLP-I polypeptide can be 10 a signal peptide sequence and/or a leader sequence. The heterologous signal sequence can be derived from a secreted human alkaline phosphatase (SEAP) signal peptide sequence, a proexendin-4 leader sequence, a pro-helodermin leader sequence, a pro-glucose dependent insulinotropic polypeptide (GIP) leader sequence, a pro-insulin growth factor 1 (IGF1) leader sequence, a preproglucagon leader sequence, an alpha-1 antitrypsin leader sequence, an insulin like growth factor 1, a human lymphotoxin A leader sequence, or a human clusterin leader sequence. In a particular embodiment, the precursor GLP-1 comprises a furin cleavage site (e.g., Arg-X-Lys-Arg (SEQ ID NO: 34), Arg-X-Arg-Arg (SEQ ID NO: 35), Lys/Arg-Arg (SEQ ID NO: 36) and Arg-X-X-Arg (SEQ ID NO: 37) such as an Arg-Gln-Lys-Arg (SEQ ID NO: 38)). In another embodiment, the precursor GLP-1 comprises a prohormone convertase (PC) cleavage site. In particular embodiments, the isolated precursor glucagon-like peptide 1 (GLP-1) comprising mammalian GLP-1 linked to a heterologous signal sequence has an amino acid sequence selected from the group consisting of SEQ ID NO: 2; SEQ ID NO: 4; SEQ ID NO: 6; SEQ ID NO: 8; SEQ ID NO: 10; SEQ ID NO: 12; SEQ ID NO: 14; SEQ ID NO: 16; SEQ ID NO: 18; and SEQ ID NO: 20.

The present invention also relates to an expression vector comprising a nucleic acid which encodes a precursor GLP-1 comprising mammalian GLP-1 linked to a heterologous signal sequence. In one embodiment, the expression vector can be a viral vector (e.g., an adenovirus vector, a partially-deleted adenovirus vector, a fully-deleted adenovirus vector, an adeno-associated virus vector, a pseudoadenovirus, a retrovirus vector, a herpesvirus and a lentivirus vector).

The present invention also relates to an isolated host cell comprising a nucleic acid which encodes a precursor GLP-1 comprising mammalian GLP-I linked to a heterologous signal sequence. In a particular embodiment, the isolated host cell comprises an expression vector described herein. The present invention also relates to a method of promoting insulin production in an individual in need thereof (e.g., Type I diabetic, Type II diabetic), comprising administering to the individual an effective amount of a nucleic acid encoding a precursor GLP-1 comprising mammalian GLP-1 linked to a heterologous signal sequence, wherein the precursor GLP-1 is cleaved in vivo or ex vivo which results in generation of activated GLP-1 in the individual. In one embodiment, the nucleic acid is administered in a viral vector (e.g., an adenovirus vector, a partially-deleted adenovirus vector, a fully-deleted adenovirus vector, an adeno-associated virus vector, a pseudoadenovirus, a retrovirus vector, a herpesvirus vector and a lentivirus vector).

The present invention also relates to a method of treating an individual having a blood sugar defect, comprising administering to the individual an effective amount of a nucleic acid encoding a precursor GLP-1 comprising mammalian GLP-1 linked to a heterologous signal sequence, wherein the precursor GLP-1 is cleaved in vivo or ex vivo which results in generation of activated GLP-1 in the individual. The blood sugar defect can be, for example, a defect such as Type I diabetes, Type II diabetes and/or hyperglycemia.

The methods of promoting insulin production and treating a blood sugar defect in an individual can also comprise administering a precursor GLP-1 peptide. In this embodiment, the protease that cleaves the heterologous signal sequence from the GLP-1 can also be administered with (simultaneously, sequentially) the GLP-1 precursor peptide.

Thus, the compositions of the present invention can be used as an alternative, effective treatment of blood sugar disorders such as diabetes.

### BRIEF DESCRIPTION OF THE DRAWINGS

FIG. 1 shows the nucleotide (SEQ ID NO: 1) and amino acid (SEQ ID NO: 2) sequences of the signal peptide from secreted human alkaline phosphatase (SEAP) linked to Gly-8 modified human GLP-1 (GLP-1-Gly8) designated SEAP.GLP-1Gly8.

FIG. 2 shows the nucleotide (SEQ ID NO: 3) and amino acid (SEQ ID NO: 4) sequences of the leader from proexendin-4 linked to GLP-1-GIy-8, designated Exendin-4.GLP-1Gly8.

FIG. 3 shows the nucleotide (SEQ ID NO: 5) and amino acid (SEQ ID NO: 6) sequences of the leader from pro-helodermin linked to GLP-1-Gly-8, designated Helodermin.GLP-1 Gly8.

FIG. 4 shows the nucleotide (SEQ ID NO: 7) and amino acid (SEQ ID NO: 8) sequences of the leader from pro-glucose dependent insulinotropic polypeptide (GIP) linked to GLP-1-Gly-8, designated GIP.GLP-IGly8.

FIG. 5 shows the nucleotide (SEQ ID NO: 9) and amino acid (SEQ ID NO: 10) sequences of the leader from pro-insulin-like growth factor 1 (IGF1) linked to GLP-1-Gly-8 via a consensus furin cleavage site, designated IGF-1 (furin).GLP-1Gly8.

FIG. 6 shows the nucleotide (SEQ ID NO: 11) and amino acid (SEQ ID NO: 12) sequences of the leader from pro-insulin-like growth factor 1 (IGF1) linked to GLP-1-Gly-8, designated IGF-1.GLP-1Gly8.

FIG. 7 shows the nucleotide (SEQ ID NO: 13) and amino acid (SEQ ID NO: 14) sequences of the leader from preproglucagon linked to GLP-1-Gly-8, designated Preproglucagon.GLP-1Gly8.

FIG. 8 shows the nucleotide (SEQ ID NO: 15) and amino acid (SEQ ID NO: 16) sequences of the leader from alpha-1 antitrypsin linked to GLP-1-Gly-8, designated Alpha-1 antitrypsin.GLP-1Gly8.

FIG. 9 shows the nucleotide (SEQ ID NO: 17) and amino acid (SEQ ID NO: 18) sequences of amino acids 1-46 of human factor IX which contains a signal peptide and a cleavage site for a prohormone convertase linked to GLP-1-Gly-8, designated Factor IX.GLP-1Gly8.

FIG. 10 shows the nucleotide (SEQ ID NO: 19) and amino acid (SEQ ID NO: 20) sequences of the leader from proexendin-4 linked to GLP-1-Gly-8 via the cleavage site of IGF-1, designated Exendin-4 (IGF-1).GLP-1Gly8.

FIG. 11 are schematics of the IGF-1.GLP-1Gly8, the Preproglucagon.GLP-1Gly8, the Alpha-1 antitrypsin.GLP-1Gly8, Exendin-4.GLP-1Gly8, the Exendin-4 (IGF-1).GLP-1Gly8, and the Factor IX.GLP-1Gly8.

FIG. 12 is a bar graph showing GLP-1 expression levels in the supernatant of 293 cells transfected with SEAP.GLP-1Gly8, Exendin-4.GLP-1 Gly8, Helodermin.GLP-1Gly8, GIP.GLP-1GLy8, IGF-1(furin).GLP-1Gly8 or a control (mock).

FIG. 13 is a bar graph showing GLP-1 expression levels in the supernatant of 293 cells transfected with Alpha-1 antitrypsin.GLP-1Gly8, Preproglucagon.GLP-1Gly8, IGF-1.GLP-1Gly8, Exendin-4.GLP-1Gly8 or Exendin-4 (IGF-1).GLP-1Gly8.

FIG. 14 is a bar graph showing GLP-1 secreted from C2C12 cells transfected with Exendin-4.GLP-1Gly8, Exendin-4 (IGF-1).GLP-1Gly8 or Factor IX.GLP-1Gly8.

FIG. 15 is a graph showing the plasma concentrations of GLP-1 in mice 20 transduced with GLP-1 expression plasmids by high-volume tail vein injection.

FIG. 16 is a graph showing the blood glucose levels of obese db/db mice and their lean littermates that were treated with a high volume injection of plasmid DNA coding for exendin4 GLP-1 under the control of the CMV enhancer/ubiquitin promoter.

FIG. 17 is a graph showing inducible expression of GLP-1 using the Valentis Gene Switch System.

FIGS. 18A-18B list examples of modified GLP-1.

FIG. 19 is a graph demonstrating that expression of GLP-1 lowers blood glucose in obese db/db mice.

FIG. 20 is a graph demonstrating that expression of GLP-1 prevents elevation of glycated hemoglobin in obese db/db mice.

FIG. 21 is a graph demonstrating that expression of GLP-1 normalizes blood glucose in obese db/db mice.

FIG. 22 is a graph demonstrating that expression of GLP-1 improves fasting blood glucose in obese db/db mice.

FIG. 23 is a graph demonstrating the insulinotropic effect of GLP-1 expression in obese db/db mice.

FIG. 24 is a graph demonstrating active plasma GLP-1 levels in obese db/db mice treated with a polynucleotide encoding for modified GLP-1.

FIG. 25 is a graph demonstrating that expression of GLP-1 reduces hyperglycemia in young obese db/db mice.

FIG. 26 is a graph demonstrating that expression of GLP-1 improves glucose tolerance test in young obese db/db mice.

FIG. 27 is a graph demonstrating that expression of GLP-1 lowers blood glucose in obese db/db mice with different starting levels of insulin.

FIG. 28 is a graph demonstrating that expression of GLP-1 improves fasting insulin levels in obese db/db mice.

FIG. 29 is a graph demonstrating that expression of GLP-1 normalizes blood glucose in fed Zucker Diabetic Fatty (ZDF) rats.

FIG. 30 is a graph demonstrating that expression of GLP-1 prevents elevation in fasting blood glucose in Zucker Diabetic Fatty (ZDF) rats.

FIG. 31 is a graph demonstrating the insulinotropic effect of GLP-1 expression in Zucker Diabetic Fatty (ZDF) rats.

FIG. 32 is a graph demonstrating active plasma GLP-1 levels in Zucker Diabetic Fatty (ZDF) rats treated with a polynucleotide encoding for modified GLP-1.

FIG. 33 is a graph demonstrating that expression of GLP-1 prevents elevation of glycated hemoglobin in Zucker Diabetic Fatty (ZDF) rats.

FIG. 34 is a graph demonstrating that expression of GLP-1 decreases food intake in Zucker Diabetic Fatty (ZDF) rats.

FIG. 35 is a graph demonstrating the effects of GLP-1 expression on body weight in Zucker Diabetic Fatty (ZDF) rats.

### DETAILED DESCRIPTION OF THE INVENTION

The present invention relates to an isolated nucleic acid (e.g., DNA, cDNA, RNA) which encodes a precursor glucagon-like peptide 1 (GLP-1) comprising mammalian GLP-1 linked to a heterologous signal sequence, isolated polypeptides encoded by the nucleic acids of the present invention, expression vectors and host cells comprising the nucleic acids of the present invention and methods of using the nucleic acids and polypeptides to promote insulin production in an individual in need thereof. The heterologous signal sequence is not the signal sequence normally associated (non-native) with the wild type GLP-1 precursor protein (i.e., the signal sequence(s) of the full length proglucagon) and provides for cleavage of the precursor GLP-1 by a protease. Upon cleavage of the signal sequence from the GLP-1 precursor by the protease, biologically active GLP-1 is produced.

As used herein, the term “isolated” (partially or substantially “purified”) refers to a composition that is substantially free of contaminating material from the source from which the composition is obtained.

GLP-1 (7-37) is a single chain glycoprotein which is insulinotropic and is secreted into the blood in an active form. In vitro and in vivo, GLP-1 is proteolytically inactivated by dipeptidyl peptidase IV. Diabetes patients appear to have reduced levels of GLP-1. As used herein, “GLP-1” means mammalian (e.g., human) GLP-1 (7-37). By custom in the art, the amino-terminus of GLP-1(7-37) has been assigned number 7 and the carboxy-terminus, number 37. The amino acid sequence of GLP-1(7-37), which is well-known in the art, is presented below:

(SEQ ID NO: 21)

NH₂-His⁷-Ala-Glu-Gly¹⁰-Thr-Phe-Thr-Ser-Asp¹⁵-
Val-Ser-Ser-Tyr-Leu²⁰-Glu-Gly-Gln-Ala-Ala²⁵-Lys-Glu-
Phe-Ile-Ala³⁰-Trp-Leu-Val-Lys-Gly³⁵-Arg-Gly³⁷⁻
COOH.

The precursor GLP-1 of the present invention can also comprise a modified GLP-1. As used herein a “modified GLP-1” is defined as a GLP-1 molecule having a (one or more) modification in the GLP-1 nucleic acid sequence and/or amino acid sequence (e.g., one or more nucleic acid and/or amino acid substitutions, deletions, inversions, or additions when compared with GLP-1) and retains the biological activity of GLP-1 when cleaved from the GLP-1 precursor. As used herein, a “biological activity of GLP-1” (“biologically active GLP-1 Pt) includes one or more biological activities associated with GLP-1 (e.g., having insulinotropic activity (the ability to promote/stimulate insulin secretion); the ability to lower glucagon secretion; the ability to affect weight loss).

A modified GLP-1 can comprise a truncated GLP-1 sequence and can also include a GLP-1 beginning from amino acids 1, 2, 3, 4, 5, 6 or 7 of the full length GLP-1 peptide, and ending at amino acid 36 or 37 of the full GLP-1 peptide. Thus, the “modified GLP-1” can include the GLP-1 beginning from amino acid 1 to amino acid 37, referred to herein as “full length glucagon-like peptide 1”. In a particular embodiment, the modified GLP-1 includes a mutation of amino acid 8 from Ala to Gly (SEQ ID NO: 22). Other modified GLP-1 molecules are known in the art, and include, for example, GLP-1(7-34) (SEQ ID NO: 23), GLP-1(7-35) (SEQ ID NO: 24), GLP-1(7-36) (SEQ ID NO: 25), Val⁸-GLP-1(7-37) (SEQ ID NO: 26), Gln⁹-GLP-1(7-37) (SEQ ID NO: 27), Thr¹⁶-Lys¹⁸-GLP-1(7-37) (SEQ ID NO: 28), Lys¹⁸

GLP-1(7-37) (SEQ ID NO: 29) and D-Gln⁹-GLP-1(7-37) (SEQ ID NO: 30). Other modified GLP-1 molecules include GLP-1 (2-37) (SEQ ID NO: 31); GLP-1 (3-37) (SEQ ID NO: 32); GLP-1 (6-37) (SEQ ID NO: 33). GLP-1(7-34) (SEQ ID NO: 23) and GLP-1(7-35) (SEQ ID NO: 24) are disclosed in U.S. Pat. No. 5,118,666. These compounds are biologically active (processed) forms of GLP (e.g., having insulinotropic properties). Additional modified GLP-1 molecules are disclosed in U.S. Pat. No. 5,545,618. Modified GLP-1 molecules suitable for the practice of the invention include the active fragment that effects weight loss. Particular modified GLP-1 molecules are those that are resistant to cleavage inactivation by dipeptidyl protease IV (DPPIV).

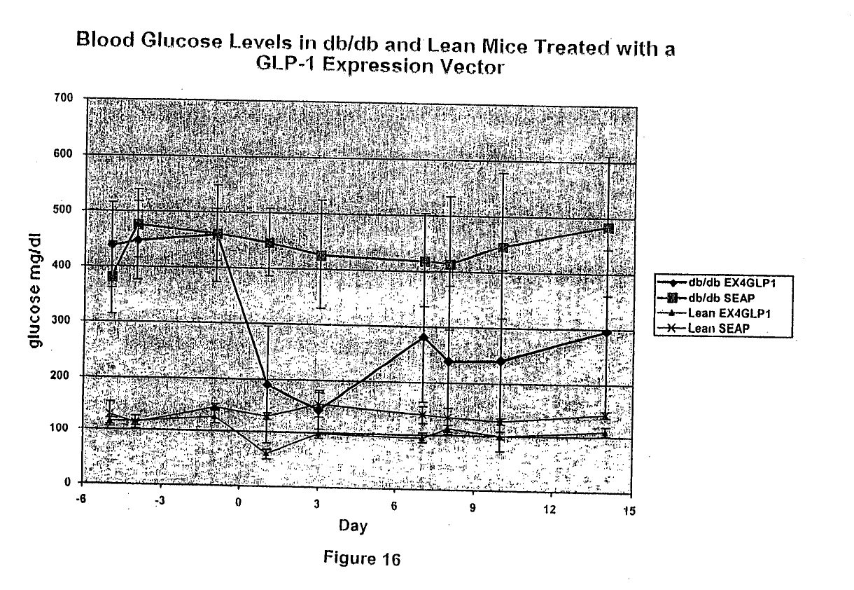

The modifications described herein can be introduced into other mammalian GLP-1 as they are identified, and whether the resulting modified GLP-1 is biologically active GLP-1 in vivo, can be assessed using known methods. The nucleic acid encoding modified GLP-1 described herein can be obtained from commercial sources, recombinantly produced or chemically synthesized. Sequence modifications of the modified GLP-1 described herein can be accomplished using a variety of techniques. For example site-directed mutagenesis and/or enzymatic cleavage can be used. Additional modified GLP-1 molecules similar to those described herein can be prepared by those of skill in the art. Such modified versions of GLP-1 can be assessed for their ability to induce the production of insulin in vivo using a variety of known assays for GLP-1 activity. For example, a modified GLP-1 can be introduced into a cell, such as an immortalized P cell, and the resulting cell can be contacted with glucose. If the cell produces insulin in response to the glucose, then the modified GLP-1 is biologically active in vivo (Fehmann, et al., Endocrinology, 130:159-166 (1992)). Alternatively, as described in Example 1 (FIG. 16), an expression plasmid comprising the modified GLP-1 can be introduced into a db/db mouse using high volume tail vein injection. If the amount of glucose in the db/db mouse is reduced upon expression of the modified GLP-1, then the modified GLP-1 is biologically active in vivo.

As used herein “a heterologous signal sequence” is defined as a signal sequence which is not the signal sequence normally associated (non-native) with the wild type GLP-1 precursor protein (i.e., the signal sequence(s) of the full length proglucagon precursor molecule) and which provides for cleavage of the precursor 15 GLP-I by a protease. Upon cleavage of the heterologous signal sequence from the GLP-1 precursor by the protease biologically active GLP-1 is produced. The heterologous signal sequence generally comprises a region which encodes a cleavage site recognized by a protease for cleavage. Alternatively, a region which encodes a cleavage site recognized by a protease for cleavage can be introduced into the heterologous signal sequence. Furthermore, additional (one or more) sequences which encodes a cleavage site recognized by a protease for cleavage can be added to the heterologous signal sequence or to the precursor GLP-1.

In a particular embodiment, the heterologous signal sequence is cleaved by a protease that is present in cells into which the precursor GLP-1 is introduced. However, if the protease that cleaves the heterologous signal sequence is not present in the cell, the protease can also be introduced into the cell into which the precursor GLP-1 has been introduced (e.g., introduced simultaneously or sequentially with the precursor GLP-1). Any suitable heterologous signal sequence which, when cleaved from the precursor GLP-1, results in generation of activated GLP-1, can be introduced into the cleavage activation site of the GLP-1 of the present invention. As used herein, the “cleavage activation site of GLP-1” is at about amino acid 7 of full length GLP-1.

A “heterologous signal sequence” can be, for example, a signal peptide sequence and/or a leader sequence (e.g., a secretory signal sequence). Examples of heterologous signal sequences which can be used in the compositions of the present invention include a signal sequence derived from a secreted protein other than GLP 1, such as a cytokine, a clotting factor, an immunoglobulin, a secretory enzyme or a hormone (including the pituitary adenylate cyclase activating polypeptide (PACAP)/glucagon superfamily) and a serum protein. For example, a heterologous signal sequence for use in the present invention can be derived from secreted human alkaline phosphatase (SEAP), pro-exendin, pro-helodermin, pro-glucose-dependent insulinotropic polypeptide (GIP), pro-insulin-like growth factor (IGF1), preproglucagon, alpha-1 antitrypsin, insulin-like growth factor 1, human factor IX, human lymphotoxin A (Genbank Accession no. BAA00064), or human clusterin (Genbank Accession no. AAP88927). Particular examples of a heterologous signal sequences are sequences which include a coding region for a signal for precursor cleavage by signal peptidase and furin or other prohormone convertase (e.g., PC3). For example, a signal which is cleaved by furin (also known as PACE, see U.S. Pat. No. 5,460,950), other subtilisins (including PC2, PC1/PC3, PACE4, PC4, PC5/PC6, LPC/PC7IPC8/SPC7 and SKI-1; Nakayama, Biochem. J., 327:625-635 (1997)); enterokinase (see U.S. Pat. No. 5,270,181) or chymotrypsin can be introduced into the cleavage activation site of GLP-1 for use in the present invention. The disclosure of each of the above documents is hereby incorporated herein by reference. Furin is a ubiquitously expressed protease that resides in the trans-golgi and processes protein precursors before their secretion. Furin cleaves at the COOH-terminus of its consensus recognition sequence, Arg-X-Lys-Arg (SEQ ID NO: 34) or Arg-X-Arg-Arg (SEQ ID NO: 35), (Lys/Arg)-Arg-X-(Lys/Arg)-Arg (SEQ ID NO: 36) and Arg-X-X-Arg (SEQ ID NO: 37), such as an Arg-Gln-Lys-Arg (SEQ ID NO:38). These amino acid sequences are a signal for precursor cleavage by the protease furin. Thus, a heterologous signal sequence can also be synthetically derived from a consensus sequence compiled from signal sequences (e.g., a consensus sequence compiled from secreted proteins that are cleaved by signal peptidase).

In particular embodiments, the heterologous signal sequence has a signal which is cleaved by a protease that is specific to a particular cell or tissue (e.g., muscle, brain). In one embodiment, the heterologous signal sequence is derived from insulin-like growth factor 1 (IGF-1) which is cleaved by a protease that is present in muscle. In addition, such heterologous signal sequences can be used with regulatory elements (e.g., promoters, enhancers) that are specific to the tissue type (e.g., muscle, liver).

Examples of such regulatory elements for muscle are provided in Souza et al., Molec. Ther., 5(5) part 2:S409 (June 2002). Muscle-specific promoters useful in the invention include mammalian muscle creatine kinase (MCK) promoter, mammalian desmin promoter, mammalian troponin I (TNNI2) promoter, or mammalian skeletal alpha-actin (ASKA) promoter. In one embodiment, the promoter is a human promoter. In another embodiment, the promoter is a murine promoter. In certain embodiments, the promoter is truncated. Tissue-specific enhancers useful in the present invention are selected from the group consisting of mammalian MCK enhancer, mammalian DES enhancer, and vertebrate troponin I IRE (TNI IRE, herein after referred to as FIRE) enhancer. One or more of these muscle-specific enhancer elements may be used in combination with a muscle-specific promoter of the invention to provide a tissue-specific regulatory element. In one embodiment, the enhancers are derived from human or mouse. In another embodiment, the FIRE enhancer is a quail promoter. In certain embodiments, the enhancer/enhancer or enhancer/promoter combinations are heterologous. In other embodiments, the enhancers and promoters are derived from the same species. In certain embodiments, the enhancer elements are truncated.

Examples of such regulatory elements for the liver are provided in WO 01/36620. Liver-specific regulatory elements may comprise strong constitutive promoters and one or more liver-specific enhancer elements. The strong constitutive promoter may be selected from the group comprising a CMV promoter, a truncated CMV promoter, a human serum albumin promoter, and an alpha-1-anti trypsin promoter. The liver-specific enhancer elements are selected from the group comprising human serum albumin [HSA] enhancers, human prothrombin [HPrT] enhancers, alpha-1 microglobulin [A1MB] enhancers, and intronic aldolase enhancers. One or more of these liver-specific enhancer elements may be used in combination with the promoter. In one embodiment, one or more HSA enhancers are used in combination with a promoter selected from the group of a CMV promoter or an HSA promoter. In another embodiment, one or more enhancer elements selected from the group consisting of human prothrombin [HPrT] and alpha-1 microglobulin [A1MB] are used in combination with the CMV promoter. In another embodiment, the enhancer elements are selected from the group consisting of human prothrombin [HPrT] and alpha-1 microglobulin [A1MB] and are used in combination with the alpha-1-anti trypsin promoter.

Furthermore, the sequences of the signal sequences can be further modified (e.g., to optimize cleavage of the precursor GLP-1). For example, the region of the IGF-1 signal sequence involved in protease cleavage can be modified as follows:

Wild type (WT)

| | |
|---|---|
| PLKPAKSAR | (SEQ ID NO: 39) |
| PLKPAKSKR | (SEQ ID NO: 40) |
| PLKPARSAR | (SEQ ID NO: 41) |
| PLRPAKSAR | (SEQ ID NO: 42) |
| PLAPAKSAR | (SEQ ID NO: 43) |
| PLKPARSKR | (SEQ ID NO: 44) |
| PLRPAKSKR | (SEQ ID NO: 45) |
| PLRPARSKR | (SEQ ID NO: 46) |
| PLAPAKSKR | (SEQ ID NO: 47) |
| PLAPARSKR | (SEQ ID NO: 48) |
| PLAPARSAR | (SEQ ID NO: 49) |
| PLRPARSAR | (SEQ ID NO: 50) |

The heterologous signal sequence is linked to GLP-1 using a variety of techniques. For example, the heterologous signal sequence can be fused in-frame at about amino acid 7 of GLP-1 using recombinant techniques resulting in production of a precursor GLP-1 which is a fusion protein. Generally, the heterologous signal sequence is fused to the N-terminus of GLP-1. For example, the heterologous signal sequence can be linked at about amino acid 6 to about amino acid 7 of GLP-1 to create an appropriate cleavage site using recombinant techniques.

In particular embodiments, the isolated nucleic acid which encodes a 5 precursor GLP-1 comprising mammalian GLP-1 linked to a heterologous signal sequence is selected from the group consisting of SEQ ID NO: 1; SEQ ID NO: 3; SEQ ID NO: 5; SEQ ID NO: 7; SEQ ID NO: 9; SEQ ID NO: 11; SEQ ID NO: 13; SEQ ID NO: 15; SEQ ID NO: 17; and SEQ ID NO: 19.

The present invention also relates to an isolated polypeptide encoded by a 10 nucleic acid described herein. For example, the isolated nucleic acid which encodes a precursor GLP-1 comprising mammalian GLP-1 linked to a heterologous signal sequence, has an amino acid sequence selected from the group consisting of: SEQ ID NO: 2; SEQ ID NO: 4; SEQ ID NO: 6;SEQ ID NO: 8; SEQ ID NO: 10; SEQ ID NO: 12; SEQ ID NO: 14; SEQ ID NO: 16;SEQ ID NO: 18; and SEQ ID NO: 20.

The precursor GLP-1 of the present invention can further comprise a component that regulates secretion of GLP-1. For example, the RPD Regulated Secretion/Aggregation Kit (Ariad Pharmaceuticals, Inc.) can be linked to the precursor GLP-I to regulate secretion of GLP-1.

The nucleic acid and amino acid sequences of the present invention can be 20 recombinantly produced, chemically synthesized or obtained from commercial sources. The nucleic acid and amino acid GLP-1 sequences and heterologous signal sequences for use in the present invention can be derived from any suitable source (e.g., mammalian) and modified as described herein. For example, the GLP-1 and heterologous signal sequence can be of human origin (U.S. Pat. Nos. 6,191,102; 5,981,488) or of bovine, murine, simian origin as well as other species' origin, and may be chimeric, for example including domains of human and non-human GLP-1 (see, for example, by analogy U.S. Pat. Nos. 5,364,771 and 5,563,045 (F VIII)).

The present invention further encompasses compositions comprising vectors 30 (e.g., expression vectors) encoding a precursor GLP-1 of the present invention. The expression vector can comprise regulatory elements which direct expression of the precursor GLP-1. In one embodiment, the precursor GLP-1 comprises an amino acid sequence which includes a coding region for a signal for precursor cleavage by furin and/or signal peptidase (e.g., the nucleic acid sequence encodes an amino acid sequence which includes a signal for precursor cleavage by furin at the activation cleavage site of the precursor GLP-1 peptide). In another embodiment, the nucleic acid sequence encodes an amino acid sequence for a signal peptide which is cleaved by signal peptidase at the activation cleavage site of the precursor GLP-1 peptide. In another embodiment, the precursor GLP-1 comprises a heterologous signal sequence which includes a coding region for a signal for precursor cleavage by signal peptidase and farm or other prohormone convertase (e.g., PC 3). In another embodiment, the nucleic acid construct comprises one or more expression constructs which encode a precursor GLP-1 and a protease that recognizes the cleavage site of the precursor GLP-1 (e.g., prohormone convertase 3 (PC3)) such that co expression in a cell yields biologically active GLP-1. In particular embodiments, the expression vector can be a viral vector (e.g., an adenovirus vector, a partially-deleted adenovirus vector, a fully-deleted adenovirus vector, an adeno-associated virus vector, a pseudoadenovirus, a retrovirus vector, a herpesvirus vector and a lentivirus vector). The present invention also relates to an isolated host cell comprising a nucleic acid which encodes a precursor GLP-1 comprising mammalian GLP-1 linked 20 to a heterologous signal sequence. In a particular embodiment, the isolated host cell comprises an expression vector described herein.

The precursor GLP-1 expressed in the host cells of the present invention is cleaved resulting in generation of biologically active GLP-1 peptide in vivo. In one embodiment, the nucleic acid sequence encodes an amino acid sequence which includes a signal for precursor cleavage by furin and/or signal peptidase at the activation cleavage site of the modified GLP-1 peptide.

Host cells comprising a nucleic acid vector encoding a precursor GLP-1 peptide in accordance with the present invention may be cultured ex vivo and administered to or implanted into an individual suffering from a diabetic disorder or disease such as type II diabetes or hyperglycemia, or insulin deficiency. The host cell comprising a nucleic acid which encodes a precursor GLP-1 comprising mammalian GLP-1 linked to a heterologous signal sequence can also be used to produce the GLP-1 precursor of the present invention. The host cell is cultured under conditions in which the precursor GLP-1 is produced. The host cell may or may not further comprise the protease that cleaves the precursor GLP-1.

The compositions of the present invention provides methods of stimulating insulin production in a cell or in an individual in need thereof, and therefore, provide alternative treatments for blood sugar disorders such as diabetes (e.g., type I diabetes, type II diabetes, hyperglycemia, insulin deficiency) in an individual.

As discussed herein, the present invention relates to nucleic acid sequences, 10 amino acid sequence and expression vectors and constructs, which provide an effective amount of biologically activate GLP-1 to, for example, the plasma, or to a suitable depot organ, such as liver or lung, of an individual in need thereof. Various embodiments of the invention are possible, each of which is capable of producing an effective amount of biologically active GLP-1 peptide in a patient who is otherwise lacking sufficient GLP-1 or insulin to achieve prandial and post-prandial glucose levels in the normal range. The present invention in various embodiments relates to methods of promoting insulin production or treating blood glucose disorders in an individual in need thereof comprising (1) administering nucleic acid which encodes a GLP-1 (2) administering nucleic acid which encodes a precursor GLP-1 comprising GLP-1 that is cleaved to form biologically active GLP-1; and (3) administering nucleic acid which encodes a precursor GLP-1 comprising a modified GLP-1 that is cleaved to form biologically active GLP-1 to the individual.

In particular, the present invention relates to a method of promoting insulin production in an individual in need thereof (e.g., Type I diabetic, Type II diabetic), comprising administering to the individual an effective amount of a nucleic acid encoding a precursor glucagon-like peptide 1 (GLP-1) comprising mammalian GLP 1 linked to a heterologous signal sequence. The precursor GLP-1 is cleaved in vivo or ex vivo which results in generation of activated GLP-1 in the individual. In one embodiment, the nucleic acid is administered in a viral vector (e.g., an adenovirus vector, a partially-deleted adenovirus vector, a fully-deleted adenovirus vector, an adeno-associated virus vector, a pseudoadenovirus, a retrovirus vector and a lentivirus vector). The nucleic acid of the present invention can also be administered as naked DNA.

The present invention also relates to a method of treating an individual having a blood sugar defect, comprising administering to the individual an effective amount of a nucleic acid encoding a precursor glucagon-like peptide 1 (GLP-1) comprising mammalian GLP-1 linked to a heterologous signal sequence, wherein the precursor GLP-1 is cleaved in vivo or ex vivo which results in generation of activated GLP in the individual. The blood sugar defect can be, for example, a defect such as Type I diabetes, Type II diabetes and/or hyperglycemia.

GLP-1 promotes insulin production in a hyperglycemic individual (that is, GLP-1 signaling in an individual is dependent on elevated glucose in the individual) and promotes β cell differentiation and replication. In an individual with Type I diabetes, the β cells are attacked by the individual's immune system. However, in the early stage of Type I diabetes, β cells are still present. Thus, administration of GLP-1 to such an individual would promote β cell differentiation and replication. Accordingly, present invention relates to a method of treating Type I diabetes, particularly in the early stage of the disease, wherein the method comprises administering the precursor GLP-1 to promote cell differentiation. In particular, the precursor GLP-1 is administered prior to elimination of all the β cells in the Type I diabetic by the immune system. In this embodiment, the method of treating Type I diabetes can further comprise the use of immunosuppression (e.g., immunosuppressive drugs) to prevent the further destruction of existing β cells by the individual's immune system.

Another effect of GLP-1 is to reduce the amount of glucagon and block gluconeogenesis by the liver in an individual. In between meals, the liver produces glucose to keep glucose levels constant. If a Type I diabetic does not produce enough insulin, the liver can produce too much glucose resulting in hyperglycemia. Thus, the precursor GLP-1 of the present invention can be used to treat a Type I diabetic, for example, by reducing the risk of hyperglycemia in the Type I diabetic and/or reducing the dosage of insulin needed by a Type I diabetic. Furthermore, administration of GLP-1 to a Type I diabetic does not impose a risk of inducing hypoglycemia in the Type I diabetic because GLP-1 only functions when an individual is hyperglycemic. That is, once the Type I diabetic is no longer hyperglycemic, GLP-1 will no longer exert its insulinotropic effect. Thus, administration of precursor GLP-1 to a Type I diabetic who is being treated with insulin improves the safety of the insulin treatment by preventing hypoglycemia without causing hyperglycemia in the process.

In the methods of the present invention, the nucleic acid encoding the precursor GLP-1 comprising a GLP-1 linked to a heterologous signal sequence may be co-expressed with a nucleic acid encoding a protease that cleaves the precursor GLP (e.g., furin). In this manner, GLP-1 could be produced in cells that would not ordinarily express the protease, and thus which would not ordinarily cleave the precursor GLP-1 product to form biologically active GLP-1. For example, the nucleic acid encoding a precursor GLP-1 (e.g., a DPPIV resistant analog) may be co expressed with a nucleic acid encoding PC3. In this manner, GLP-1 could be produced in cells that would not ordinarily express PC3, and thus, which would not ordinarily cleave proglucagon to form GLP-1.

The methods of promoting insulin production or treating a blood sugar disorder can also be carried out by administering precursor GLP-1 peptide to an individual in need thereof. In one embodiment, since the precursor GLP-1 is delivered outside the cell, the protease that cleaves the precursor GLP-1 is also administered, thereby resulting in delivery of biologically active GLP-1 in vivo. In another embodiment, the precursor GLP-1 can comprise GLP-1 linked to a cleavage site recognized by a protease in the blood. The GLP-1 precursor can also be linked to a molecule (e.g., a peptide such as albumin) that makes the precursor GLP-1 more stable and/or that results in cleavage of the precursor GLP-1 in particular areas of the body (e.g., liver, muscle, brain).

The nucleic acid encoding a precursor GLP-1 may be administered in a viral vector such as an adenovirus vector, a partially-deleted adenovirus vector, a fully-deleted adenovirus vector, an adeno-associated virus vector, a pseudoadenovirus, a retrovirus vector, a herpesvirus vector and a lentivirus vector. The nucleic acid can also be administered as naked DNA.

Insulin

The immediate precursor of insulin is a single polypeptide, termed proinsulin, which contains the two insulin chains A and B connected by another peptide, C. See Steiner, D. F., Cunningham, D., Spigelman, L. and Aten, B., Science 157, 697 (1967). It has been reported that the initial translation product of insulin mRNA is not proinsulin itself, but a preproinsulin that contains more than 20 additional amino acids on the amino terminus of proinsulin, See Calm et al. PNAS USA 73, 1964 (1976) and Lomedico and Saunders, Nucl. Acids Res. 3, 381 (1976). The structure of the preproinsulin molecule can be represented schematically as NH.sub.2-(pre-peptide)-B chain-(C peptide)-A chain-COOH. Preproinsulin is processed to mature insulin at the B/C and C/A junctions by specific proteases, PC2 and PC3, in pancreatic islet β-cells. To allow proinsulin processing to occur in a wide variety of cell types, many groups have modified rat and human insulins so that they can be processed in the constitutive pathway of secretion. It has been shown that introduction of furin consensus cleavage sequences at the B/C and A/C junctions allows efficient processing of the modified proinsulins to mature insulin in different cell types.

Many proteins of medical or research significance are found in or made by the cells of higher organisms such as vertebrates. These include, for example, the hormone insulin, other peptide hormones such as growth hormone, proteins involved in the regulation of blood pressure, and a variety of enzymes having industrial, medical or research significance. It is frequently difficult to obtain such proteins in usable quantities by extraction from the organism, and this problem is especially acute in the case of proteins of human origin. Therefore there is a need for techniques whereby such proteins can be made by cells outside the organism in reasonable quantity. In certain instances, it is possible to obtain appropriate cell lines which can be maintained by the techniques of tissue culture. However, the growth of cells in tissue culture is slow, the medium is expensive, conditions must be accurately controlled, and yields are low. Moreover, it is often difficult to maintain a cultured cell line having the desired differentiated characteristics. [U.S. Pat. No. 4,652,525].

Thus, in certain embodiments the present invention comprises methods of treating glucose disorders by administering a nucleic acid which encodes an insulin or modified insulin, together with regulatory elements which will provide for expression of the coding sequence.

In preferred embodiments, the regulatory elements will be inducible; most preferably, the regulatory elements will be responsive to insulin and/or glucose. Thus, the preferred regulatory elements include those promoters and/or enhancers described below.

In certain preferred embodiments, the present invention comprises methods 10 of treating glucose disorders by administering a nucleic acid which encodes an insulin or modified insulin, together with regulatory elements which will provide for expression of the coding sequence, and a nucleic acid which encodes a precursor GLP-1 of the present invention, together with regulatory elements which will provide for expression of the coding sequence.

Optionally, it may be advantageous to co-administer C-peptide, or a gene therapy vector encoding C-peptide, along with the insulin and/or GLP-I vectors of the present invention.

Thus, the present invention in various embodiments thus comprises (1) administering nucleic acid which encodes insulin; (2) administering nucleic acid which encodes a modified insulin that will exhibit activity similar to insulin; (3) administering nucleic acid which encodes (a) precursor (GLP-1 of the present invention together with (b) insulin or a modified insulin; and (4) administering nucleic acid which encodes (a) precursor GLP-1 of the present invention together with (b) insulin or a modified insulin together with peptide C or a DNA vector encoding peptide C.

Accordingly, the present invention provides methods of treatment of patients suffering from blood sugar disorders, such as hyperglycaemia, hypoglycaemia, diabetes type I, diabetes type II, and hypoinsulinism. In certain embodiments, the invention provides materials and methods for the treatment of blood sugar disorders, using vectors which provide insulin to the patient. In other embodiments, the invention provides materials and methods for the treatment of blood sugar disorders using vectors which provide GLP-1 to the patient. In certain preferred embodiments, both insulin and GLP-1 may be provided to the patient. In certain embodiments of the invention, vectors are provided which comprise regulatory elements, such as promoters and enhancers that may be controlled by the levels of insulin, glucose or other biological and chemical factors in the bloodstream of the patient.

In certain methods of the present invention, GLP-1, or modified GLP-1, is provided to a patient suffering from a glucose utilizing disorder, such as diabetes. The GLP-1 can be delivered via DNA vectors, which may be viral or non-viral in origin. In one embodiment, the GLP-1 is provided using a DNA vector encoding a modified GLP-1 peptide.

In other embodiments, the present invention relates to methods of treating an individual having a diabetic disorder or a hyperglycemic disorder, comprising administering to the individual an effective amount of a DNA vector expressing GLP-1 or modified GLP-1 in vivo with the result being normalization of blood glucose levels, and over time, reduction of glycated hemoglobin levels (HB1_{AC}).

Administration

Nucleic acid encoding precursor GLP-I of the present invention can be administered as any gene transfer vector, such as viral vectors, including adenovirus, AAV, retrovirus and lentivirus, as well as plasmid DNA with or without a suitable lipid or polymer carriers, and is administered under conditions in which the nucleic acid is expressed in vivo. Alternatively, nucleic acid encoding precursor GLP-1 of the present invention can be administered as naked DNA or in association with an amphiphilic compound, such as lipids or compounds, or with another suitable carrier.

The precursor GLP-1 can also be delivered as a peptide along with (simultaneously, sequentially) with the protease that cleaves the precursor GLP-1. The precursor GLP-1 and/or insulin of the present invention can be administered by introducing nucleic acid (e.g., DNA, cDNA, RNA) encoding the precursor GLP-I and/or insulin into the individual wherein the nucleic acid is expressed and biologically active GLP-1 and/or insulin is generated in vivo. Alternatively, the nucleic acid encoding the precursor GLP-1 and/or insulin can be administered ex vivo to cells (e.g., hepatocytes, myoblasts, fibroblasts, endothelial cells, keratinocytes, hematopoietic cells) of the individual and then transferred into the individual wherein the precursor GLP-I and/or insulin is expressed and biologically active GLP-1 and/or insulin is generated in vivo.

The nucleic acid (e.g., cDNA) encoding precursor GLP-1 and/or insulin can be cloned into an expression cassette that has a regulatory element such as a promoter (constitutive or regulatable) to drive transgene expression and a polyadenylation sequence downstream of the nucleic acid. For example, regulatory elements that are 1) specific to a tissue or region of the body; 2) constitutive; 3) glucose responsive; and/or 4) inducible/regulatable can be used. Suitable promoters include the cytomegalovirus (CMV) promoter, the CMV enhancer linked to the ubiquitin promoter (Cubi). Muscle-specific regulatory elements include muscle-specific promoters including mammalian muscle creatine kinase (MCK) promoter, mammalian desmin promoter, mammalian troponin I (TNNI2) promoter, or mammalian skeletal alpha-actin (ASKA) promoter. Muscle-specific enhancers useful in the present invention are selected from the group consisting of mammalian MCK enhancer, mammalian DES enhancer, and vertebrate troponin I IRE (TNI IRE, herein after referred to as FIRE) enhancer. One or more of these muscle-specific enhancer elements may be used in combination with a muscle-specific promoter of the invention to provide a tissue-specific regulatory element. Liver-specific regulatory elements may comprise strong constitutive promoters and one or more liver-specific enhancer elements. The strong constitutive promoter may be selected from the group comprising a CMV promoter, a truncated CMV promoter, a human serum albumin promoter, and an alpha-1-anti trypsin promoter. The liver-specific enhancer elements are selected from the group comprising human serum albumin [HSA] enhancers, human prothrombin [HPrT] enhancers, alpha-1 microglobulin [A1MB] enhancers, and intronic aldolase enhancers. One or more of these liver-specific enhancer elements may be used in combination with the promoter. In one embodiment, one or more HSA enhancers are used in combination with a promoter selected from the group of a CMV promoter or an HSA promoter. In another embodiment, one or more enhancer elements selected from the group consisting of human prothrombin [HPrT] and alpha-1 microglobulin [A1MB] are used in combination with the CMV promoter. In another embodiment, the enhancer elements are selected from the group consisting of human prothrombin [HPrT] and alpha-1 microglobulin [A1MB] and are used in combination with the alpha-1-anti trypsin promoter. Conditional promoters such as the dimerizer gene control system, based on the immunosuppressive agents FK506 and rapamycin, the ecdysone gene control system and the tetracycline gene control system. Also useful in the present invention are regulatory sequences which can regulate transcription of the precursor GLP-1 of the present invention, such as the GeneSwitch™ technology (Valentis, Inc., Woodlands, Tex.) described in Abruzzese et al., Hum. Gene Ther. 1999 10: 1499-507, the disclosure of which is hereby incorporated herein by reference. With inducible or regulatable promoters, the clinician may exert additional optimization of the methods of the present invention, such that optimal levels of biologically active GLP-1 and/or insulin are achieved for blood sugar control.

Particular promoters are of human or mammalian origin. The promoter sequence may be a constitutive promoter, or maybe an inducible promoter. In preferred embodiments the promoter may be inducible. Particularly preferred promoter sequences for use in the present invention include liver type pyruvate kinase promoters, particularly those fragments which run (−183 to +12) or (−96 to +12) (Thompson, et al. J Biol Chem, (1991). 266:8679-82.; Cuif, et al., Mol Cell Biol, (1992). 12:4852-61); the spot 14 promoter (S14, −290 to +18) (Jump, et al., J. Biol Chem, (1990). 265:3474-8); acetyl-CoA carboxylase (O'Callaghan, et al., J. Biol Chem, (2001). 276:16033-9); fatty acid synthase (−600 to +65) (Rufo, et al., J Biol Chem, (2001). 28:28); and glucose-6-phosphatase (rat and human) (Schmoll, et al., FEBS Left, (1996). 383:63-6; Argaud, et al., Diabetes, (1996). 45:1563-71).

In particular embodiments of the present invention, the insulin coding 5 sequence or GLP-1 coding sequence is further under the control of one or more enhancer elements. Among those enhancer elements which will be most useful in the present invention are those which are glucose responsive, insulin responsive and/or liver specific. Particular embodiments may include the CMV enhancer (e.g., linked to the ubiquitin promoter (Cubi)); one or more glucose responsive elements, including the glucose responsive element (G1RE) of the liver pyruvate kinase (L-PK) promoter (−172 to −142); and modified versions with enhanced responsiveness (Cuif et al., supra; Lou, et al., J. Biol Chem, (1999). 274:28385-94); G1RE of L-PK with auxiliary L3 box (−172 to −126) (Diaz Guerra, et al., Mol Cell Biol, (1993). 13:7725-33; modified versions of G1RE with enhanced responsiveness with the auxiliary L3 box; carbohydrate responsive element (ChoRE) of S 14 (−1448 to −1422), and modifications activated at lower glucose concentrations (Shih and Towle, J Biol Chem, (1994). 269:9380-7; Shih, et al., J Biol Chem, (1995). 270:21991-7; and Kaytor, et al., J Biol Chem, (1997). 272:7525-31; ChoRE with adjacent accessory factor site of S 14 (−1467 to −1422) [et al., supra]; aldolase (+1916 to +2329)(Gregori et al., J Biol Chem, (1998). 273:25237-43; Sabourin, et al., J. Biol Chem, (1996). 271:3469-73; and fatty acid synthase (−7382 to −6970) (Rufo, et al., supra.). Preferred embodiments may also include insulin responsive elements such as glucose-6-phosphatase insulin responsive element (−780 to −722) [Ayala et al., Diabetes, (1999). 48:1885-9; and liver specific enhancer elements, such as prothrombin (940 to −860) [Chow et al., J Biol Chem, (1991) 266: 18927-33; and alpha-1-microglobulin (−2945 to −2539) [Rouet et al., Biochem J, (1998). 334:577-84).

The expression cassette is then inserted into a vector such as adenovirus, partially-deleted adenovirus, fully-deleted adenovirus, adeno-associated virus (AAV), retrovirus, lentivirus, naked plasmid, plasmid/liposome complex, etc. for delivery to the host via intravenous, intramuscular, intraportal or other route of administration. Expression vectors which can be used in the methods and compositions of the present invention include, for example, viral vectors. One of the most frequently used methods of administration of gene therapy, both in vivo and ex vivo, is the use of viral vectors for delivery of the gene. Many species of virus are known, and many have been studied for gene therapy purposes. The most commonly used viral vectors include those derived from adenoviruses, adeno associated viruses (AAV) and retroviruses, including lentiviruses, such as human immunodeficiency virus (HIV).

Adenoviral vectors for use to deliver transgenes to cells for applications such 10 as in vivo gene therapy and in vitro study and/or production of the products of transgenes, commonly are derived from adenoviruses by deletion of the early region 1 (El) genes (Berkner, K. L., Curr. Top. Micro. Immunol. 158 L39-66 1992). Deletion of El genes renders such adenoviral vectors replication defective and significantly reduces expression of the remaining viral genes present within the vector. However, it is believed that the presence of the remaining viral genes in adenoviral vectors can be deleterious to the transfected cell for one or more of the following reasons: (1) stimulation of a cellular immune response directed against expressed viral proteins, (2) cytotoxicity of expressed viral proteins, and (3) replication of the vector genome leading to cell death.

One solution to this problem has been the creation of adenoviral vectors with deletions of various adenoviral gene sequences. In particular, pseudoadenoviral vectors (PAVs), also known as ‘gutless adenovirus’ or mini-adenoviral vectors, are adenoviral vectors derived from the genome of an adenovirus that contain minimal cis-acting nucleotide sequences required for the replication and packaging of the vector genome and which can contain one or more transgenes (See, U.S. Pat. No. 5,882,877 which covers pseudoadenoviral vectors (PAV) and methods for producing PAV, incorporated herein by reference). Such PAVs, which can accommodate up to about 36 kb of foreign nucleic acid, are advantageous because the carrying capacity of the vector is optimized, while the potential for host immune responses to the vector or the generation of replication-competent viruses is reduced. PAV vectors contain the 5′ inverted terminal repeat (ITR) and the 3′ITR nucleotide sequences that contain the origin of replication, and the cis-acting nucleotide sequence required for packaging of the PAV genome, and can accommodate one or more transgenes with appropriate regulatory elements, e.g. promoter, enhancers, etc.

Other, partially deleted adenoviral vectors provide a partially-deleted 5 adenoviral (termed “DeAd”) vector in which the majority of adenoviral early genes required for virus replication are deleted from the vector and placed within a producer cell chromosome under the control of a conditional promoter. The deletable adenoviral genes that are placed in the producer cell may include E1A/E1B, E2, E4 (only ORF6 and ORF6/7 need be placed into the cell), pIX and pIVa2. E3 may also be deleted from the vector, but since it is not required for vector production, it can be omitted from the producer cell. The adenoviral late genes, normally under the control of the major late promoter (MLP), are present in the vector, but the MLP may be replaced by a conditional promoter.

Conditional promoters suitable for use in DeAd vectors and producer cell lines include those with the following characteristics: low basal expression in the uninduced state, such that cytotoxic or cytostatic adenovirus genes are not expressed at levels harmful to the cell; and high level expression in the induced state, such that sufficient amounts of viral proteins are produced to support vector replication and assembly. Preferred conditional promoters suitable for use in DeAd vectors and producer cell lines include the dimerizer gene control system, based on the immunosuppressive agents FK506 and rapamycin, the ecdysone gene control system and the tetracycline gene control system. Also useful in the present invention may be the GeneSwitch™ technology [Valentis Inc., Woodlands, Tex.] described in Abruzzese et al., Hum. Gene Ther. 1999 10:1499-507, the disclosure of which is hereby incorporated herein by reference.

The partially deleted adenoviral expression system is further described in W099/57296, the disclosure of which is hereby incorporated by reference herein.

Adenoviral vectors, such as PAVs and DeAd vectors, have been designed to take advantage of the desirable features of adenovirus which render it a suitable vehicle for delivery of nucleic acids to recipient cells. Adenovirus is a non-enveloped, nuclear DNA virus with a genome of about 36 kb, which has been well-characterized through studies in classical genetics and molecular biology (Hurwitz, M. S., Adenoviruses Virology, 3^{rd} edition, Fields et al., eds., Raven Press, New York, 1996; Hitt, M. M. et al., Adenovirus Vectors, The Development of Human Gene Therapy, Friedman, T. ed., Cold Spring Harbor Laboratory Press, New York 1999). The viral genes are classified into early (designated E1-E4) and late (designated L1-L5) transcriptional units, referring to the generation of two temporal classes of viral proteins. The demarcation of these events is viral DNA replication. The human adenoviruses are divided into numerous serotypes (approximately 47, numbered accordingly and classified into 6 groups: A, B, C, D, E and F), based upon properties including hemaglutination of red blood cells, oncogenicity, DNA and protein amino acid compositions and homologies, and antigenic relationships.

Recombinant adenoviral vectors have several advantages for use as gene delivery vehicles, including tropism for both dividing and non-dividing cells, minimal pathogenic potential, ability to replicate to high titer for preparation of vector stocks, and the potential to carry large inserts (Berkner, K. L., Curr. Top. Micro. Immunol. 158:39-66, 1992; Jolly, D., Cancer Gene Therapy 1:51-64 1994).

PAVs have been designed to take advantage of the desirable features of adenovirus which render it a suitable vehicle for gene delivery. While adenoviral vectors can generally carry inserts of up to 8 kb in size by the deletion of regions which are dispensable for viral growth, maximal carrying capacity can be achieved with the use of adenoviral vectors containing deletions of most viral coding sequences, including PAVs. See U.S. Pat. No. 5,882,877 of Gregory et al.; Kochanek et al., Proc. Natl. Acad. Sci. USA 93:5731-5736, 1996; Parks et al., Proc. Natl. Acad. Sci. USA 93:13565-13570, 1996; Lieber et al., J. Virol. 70:8944-8960, 1996; Fisher et al., Virology 217:11-22, 1996; U.S. Pat. No. 5,670,488; PCT Publication No. W096/33280, published Oct. 24, 1996; PCT Publication No. W096/40955, published Dec. 19, 1996; PCT Publication No. W097/25446, published Jul. 19, 1997; PCT Publication No. W095/29993, published Nov. 9, 1995; PCT Publication No. W097/00326, published Jan. 3, 1997; Morral et al., Hum. Gene Ther. 10:2709-2716, 1998.

Since PAVs are deleted for most of the adenovirus genome, production of PAVs requires the furnishing of adenovirus proteins in trails which facilitate the replication and packaging of a PAV genome into viral vector particles. Most commonly, such proteins are provided by infecting a producer cell with a helper adenovirus containing the genes encoding such proteins.

However, such helper viruses are potential sources of contamination of a PAV stock during purification and can pose potential problems when administering the PAV to an individual if the contaminating helper adenovirus can replicate and be packaged into viral particles.

The use of adenoviruses for gene therapy is described, for example, in U.S. Pat. No. 5,882,877; U.S. patent, the disclosures of which are hereby incorporated herein by reference.

Adeno-associated virus (AAV) is a single-stranded human DNA parvovirus whose genome has a size of 4.6 kb. The AAV genome contains two major genes: the rep gene, which codes for the rep proteins (Rep 76, Rep 68, Rep 52, and Rep 40) and the cap gene, which codes for AAV replication, rescue, transcription and integration, while the cap proteins form the AAV viral particle. AAV derives its name from its dependence on an adenovirus or other helper virus (e.g., herpesvirus) to supply essential gene products that allow AAV to undergo a productive infection, i.e., reproduce itself in the host cell. In the absence of helper virus, AAV integrates as a provirus into the host cell's chromosome, until it is rescued by superinfection of the host cell with a helper virus, usually adenovirus (Muzyczka, Curr. Top. Micro. Immunol. 158:97-127, 1992).

Interest in AAV as a gene transfer vector results from several unique features 25 of its biology. At both ends of the AAV genome is a nucleotide sequence known as an inverted terminal repeat (ITR), which contains the cis-acting nucleotide sequences required for virus replication, rescue, packaging and integration.

There are other advantages to the use of AAV for gene transfer. The host range of AAV is broad. Moreover, unlike retroviruses, AAV can infect both quiescent and dividing cells. In addition, AAV has not been associated with human disease, obviating many of the concerns that have been raised with retrovirus derived gene transfer vectors. Recently, alternative tissue tropisms have been demonstrated for the different AAV serotype. For example Chao et al (Mol. Ther. 2000, 2:619-23) demonstrated that AAV1 when injected into skeletal muscle, can direct expression of FIX into the blood that is several logs higher than that obtained with AAV2.

Standard approaches to the generation of recombinant rAAV vectors have required the coordination of a series of intracellular events: transfection of the host cell with an rAAV vector genome containing a transgene of interest flanked by the AAV 1TR sequences, transfection of the host cell by a plasmid encoding the genes for the AAV rep and cap proteins which are required in trans, and infection of the transfected cell with a helper virus to supply the non-AAV helper functions required in trans (Muzyczka, N., Curr. Top. Micro Immunol. 158:97-129, 1992). The adenoviral (or other helper virus) proteins activate transcription of the AAV rep gene, and the rep proteins then activate transcription of the AAV cap genes. The cap proteins then utilize the ITR sequences to package the rAAV genome into an rAAV viral particle. Therefore, the efficiency of packaging is determined, in part, by the availability of adequate amounts of the structural proteins, as well as the accessibility of any cis-acting packaging sequences required in the rAAV vector genome.

Other approaches to improving the production of rAAV vectors include the use of helper virus induction of the AAV helper proteins (Clark, et al., Gene Therapy 3:1124-1132, 1996) and the generation of a cell line containing integrated copies of the rAAV vector and AAV helper genes so that infection by the helper virus initiates rAAV production (Clark et al., Human Gene Therapy 6:1329-1341, 25 1995).

rAAV vectors have been produced using replication-defective helper adenoviruses which contain the nucleotide sequences encoding the rAAV vector genome (U.S. Pat. No. 5,856,152 issued Jan. 5, 1999) or helper adenoviruses which contain the nucleotide sequences, encoding the AAV helper proteins (PCT International Publication W095/06743, published Mar. 9, 1995). Production strategies which combine high level expression of the AAV helper genes and the optimal choice of cis-acting nucleotide sequences in the rAAV vector genome have been described (PCT International Application No. W097/09441 published Mar. 13, 1997).

Current approaches to reducing contamination of rAAV vector stocks by 5 helper viruses, therefore, involve the use of temperature-sensitive helper viruses (Ensigner et al., J. Virol., 10:328-339, 1972), which are inactivated at the non-permissive temperature. Alternatively, the non-AAV helper genes can be subcloned into DNA plasmids which are transfected into a cell during rAAV vector production (Salvetti et al., Hum. Gene Ther. 9:695-706, 1998; Grimm, et al., Hum. Gene Ther. 9:2745-2760, 1998; W097/09441). The use of AAV for gene therapy is described, for example, in U.S. Pat. No. 5,753,500, the disclosures of each of the above are hereby incorporated herein by reference.

Retrovirus vectors are a common tool for gene delivery (Miller, Nature (1992) 357:455-460). The ability of retrovirus vectors to deliver an unrearranged, single copy gene into a broad range of rodent, primate and human somatic cells makes retroviral vectors well suited for transferring genes to a cell.

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. A helper virus is not required for the production of the recombinant retrovirus if the sequences for encapsidation are provided by co-transfection with appropriate vectors.

Another useful tool for producing recombinant retroviral vectors are packaging cell lines which supply in trans the proteins necessary for producing infectious virions, but those cells are incapable of packaging endogenous viral genomic nucleic acids (Watanabe & Termin, Molec. Cell. Biol. (1983) 3(12):2241-2249; Mann et al., Cell (1983) 33:153-159; Embretson & Temin, J. Virol. (1987) 61(9):2675-2683). One approach to minimize the likelihood of generating RCR in packaging cells is to divide the packaging functions into two genomes, for example, one which expresses the gag and pol gene products and the other which expresses the env gene product (Bosselman et al., Molec. Cell. Biol. (1987) 7(5):1797-1806; Markowitz et al., J. Virol. (1988) 62(4):1 120-1124; Danos & Mulligan, Proc. Natl. Acad. Sci. (1988) 85:6460-6464). That approach minimizes the ability for co packaging and subsequent transfer of the two-genomes, as well as significantly decreasing the frequency of recombination due to the presence of three retroviral genomes in the packaging cell to produce RCR.

Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. Other examples of lentiviral vectors include, feline immunodeficiency virus (FIV), simian immunodeficiency virus (SIV), equine immunodeficiency virus (EAIV) and simian foamy virus type-1 (SFV-1). In vivo, HIV can infect terminally differentiated cells that rarely divide, such as lymphocytes and macrophages. In vitro, HIV can infect primary cultures of monocyte-derived macrophages (MDM) as well as HeLa-Cd4 or T lymphoid cells arrested in the cell cycle by treatment with aphidicolin or gamma irradiation. Infection of cells is dependent on the active nuclear import of HIV preintegration complexes through the nuclear pores of the target cells. That occurs by the interaction of multiple, partly redundant, molecular determinants in the complex with the nuclear import machinery of the target cell. Identified determinants include a functional nuclear localization signal (NLS) in the gag matrix (MA) protein, the karyophilic virion-associated protein, vpr, and a C-terminal phosphotyrosine residue in the gag MA protein. The use of retroviruses for gene therapy is described, for example, in U.S. Pat. No. 6,013,516; and U.S. Pat. No. 5,994,136, the disclosures of which are hereby incorporated herein by reference.

Other methods for delivery of nucleic acid to cells do not use viruses for delivery. For example, cationic amphiphilic compounds can be used to deliver the nucleic acid of the present invention. Because compounds designed to facilitate intracellular delivery of biologically active molecules must interact with both non polar and polar environments (in or on, for example, the plasma membrane, tissue fluids, compartments within the cell, and the biologically active molecular itself), such compounds are designed typically to contain both polar and non-polar domains. Compounds having both such domains may be termed amphiphiles, and many lipids and synthetic lipids that have been disclosed for use in facilitating such intracellular delivery (whether for in vitro or in vivo application) meet this definition. One particularly important class of such amphiphiles is the cationic amphiphiles. In general, cationic amphiphiles have polar groups that are capable of being positively charged at or around physiological pH, and this property is understood in the art to be important in defining how the amphiphiles interact with the many types of biologically active (therapeutic) molecules including, for example, negatively charged polynucleotides such as DNA.

Examples of cationic amphiphilic compounds that have both polar and non polar domains and that are stated to be useful in relation to intracellular delivery of biologically active molecules are found, for example, in the following references, which contain also useful discussion of (1) the properties of such compounds that are understood in the art as making them suitable for such applications, and (2) the nature of structures, as understood in the art, that are formed by complexing of such amphiphiles with therapeutic molecules intended for intracellular delivery.

(1) Felgner, et al., Proc. Natl. Acad. Sci. USA, 84, 7413-7417 (1987) disclose use of positively-charged synthetic cationic lipids including N→1(2,3-dioleyloxy)propyl1-N,N,N-trimethylammonium chloride (“DOTMA”), to form lipid/DNA complexes suitable for transfections. See also Feigner et al., The Journal of Biological Chemistry, 269(4), 2550-2561 (1994).

(2) Behr et al., Proc. Natl. Acad. Sci USA, 86, 6982-6986 (1989) disclose numerous amphiphiles including dioctadecylamidologlycylspermine (“DOGS”).

(3) U.S. Pat. No. 5,283,185 to Epand et al. describes additional classes and 30 species of amphiphiles including 3.betaN-(N.sup.1,N.sup.1-dimethylaminoethane)carbamoyl1 cholesterol, termed “DC-chol”.

(4) Additional compounds that facilitate transport of biologically active molecules into cells are disclosed in U.S. Pat. No. 5,264,618 to Felgner et al. See also Feigner et al., The Journal of Biological Chemistry, 269(4), pp. 2550-2561 (1994) for disclosure therein of further compounds including “DMRIE” 1,2-dimyristyloxypropyl-3-dimethyl-hydroxyethyl ammonium bromide.

(5) Reference to amphiphiles suitable for intracellular delivery of biologically active molecules is also found in U.S. Pat. No. 5,334,761 to Gebeyehu et al., and in Felgner et al., Methods (Methods in Enzymology), 5, 67-75 (1993). The use of compositions comprising cationic amphiphilic compounds for gene delivery is described, for example, in U.S. Pat. No. 5,049,386; U.S. Pat. No. 5,279,833; U.S. Pat. No. 5,650,096; U.S. Pat. No. 5,747,471; U.S. Pat. No. 5,767,099; U.S. Pat. No. 5,910,487; U.S. Pat. No. 5,719,131; U.S. Pat. No. 5,840,710; U.S. Pat. No. 5,783,565; U.S. Pat. No. 5,925,628; U.S. Pat. No. 5,912,239; U.S. Pat. 5,942,634; U.S. Pat. No. 5,948,925; U.S. Pat. No. 6,022,874;U.S. Pat. No. 5,994,317; U.S. Pat. No. 5,861,397; U.S. Pat. No. 5,952,916; U.S. Pat. No. 5,948,767; U.S. Pat. No. 5,939,401; and U.S. Pat. No. 5,935,936, the disclosures of which are hereby incorporated herein by reference.

In addition, nucleic acid encoding precursor GLP-1 and/or insulin of the present invention can be delivered using “naked DNA”. Methods for delivering a non-infectious, non-integrating nucleic acid sequence encoding a desired polypeptide or peptide operably linked to a promoter, free from association with transfection-facilitating proteins, viral particles, liposomal formulations, charged lipids and calcium phosphate precipitating agents are described in U.S. Pat. No. 5,580,859; U.S. Pat. No. 5,963,622; U.S. Pat. No. 5,910,488; the disclosures of which are hereby incorporated herein by reference.

Gene transfer systems that combine viral and nonviral components have also 25 been reported. Cristiano et al., (1993) Proc. Natl. Acad. Sci. USA 90:11548; Wu et al. ,(1994) J. Biol. Chem. 269:11542; Wagner et al. (1992) Proc. Natl. Acad. Sci. USA 89:6099; Yoshimura et al., (1993) J. Biol. Chem. 268:2300; Curiel et al., (1991) Proc. Natl. Acad. Sci. USA 88:8850; Kupfer et al., (1994) Human Gene Ther. 5:1437; and Gottschalk et al., (1994) Gene Ther. 1:185. In most cases, adenovirus has been incorporated into the gene delivery systems to take advantage of its endosomolytic properties. The reported combinations of viral and nonviral components generally involve either covalent attachment of the adenovirus to a gene delivery complex or co-internalization of unbound adenovirus with cationic lipid:DNA complexes.

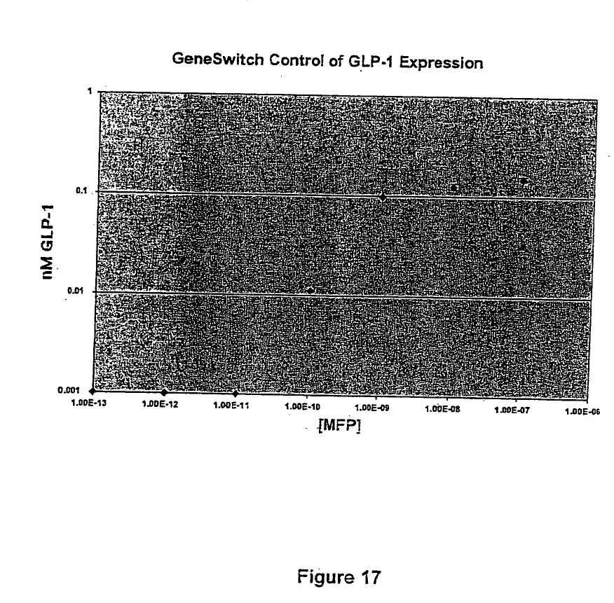

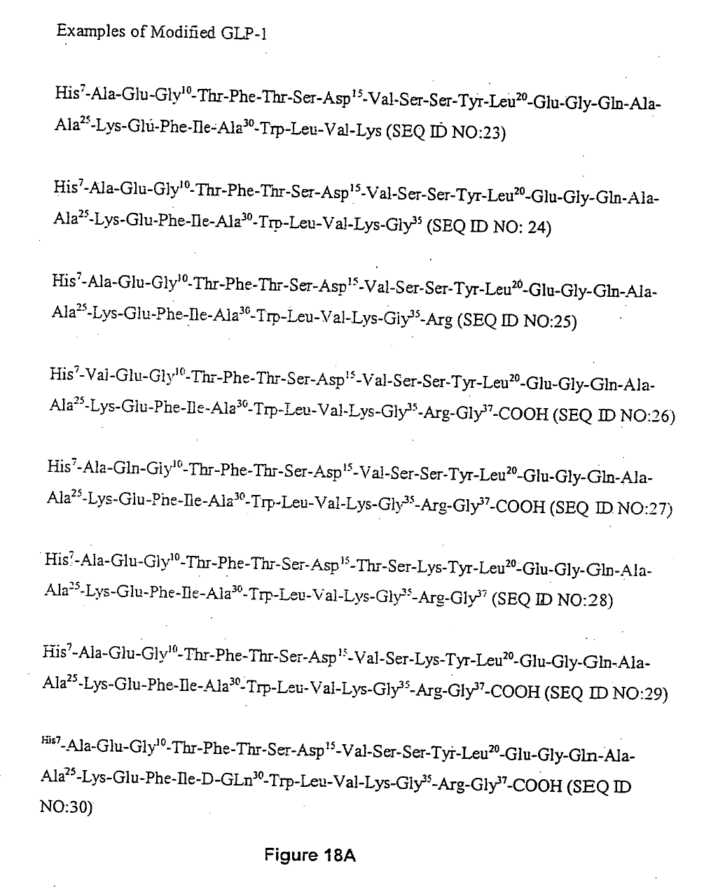

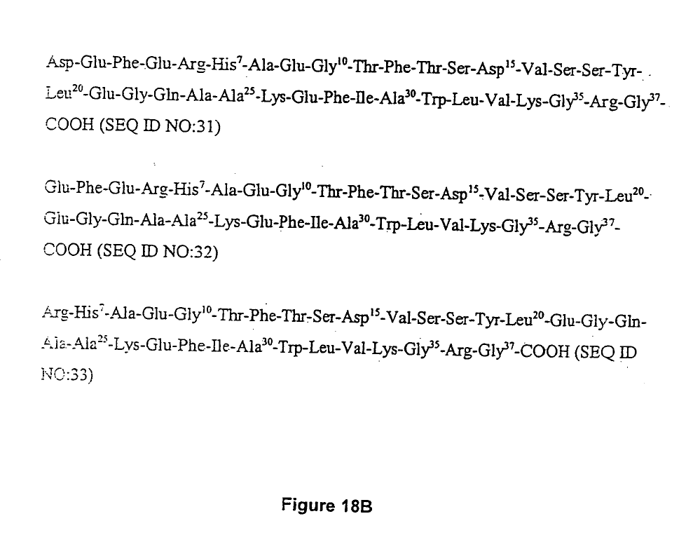

As described herein, an “effective amount” of DNA vectors encoding the insulin, modified insulin and/or precursor GLP-1 is an amount such that when administered, it produces biologically active insulin or GLP-1 molecule, which results in enhanced blood sugar or insulin levels in the individual to whom it is administered relative to blood sugar or insulin levels when an effective amount of these vectors capable of producing activated insulin or GLP-1 protein is not administered. In addition, the amount of modified insulin or GLP-1 administered to an individual will vary depending on a variety of factors, including the size, age, body weight, general health, sex and diet of the individual. In the particular embodiments wherein adenoviral or AAV vectors are used, the dose of the nucleic acid encoding precursor GLP-1 and/or insulin can be delivered via adenoviral or AAV particles, generally in the range of about 10⁶ to about 10¹⁵ particles, more preferably in the range of about 10⁸ to about 10¹³ particles. In the particular embodiments wherein retroviral or lentiviral vectors are used, the dose of the nucleic acid encoding modified insulin or precursor GLP-1 can be delivered via retroviral or lentiviral particles, generally in the range of about 10⁴ to about 10¹³ particles, more preferably in the range of about 10⁶ to about 10¹¹ particles. When nucleic acid is delivered in the form of plasmid DNA, a useful dose will generally range from about 1 ug to about 1 g of DNA, preferably in the range from about 100 ug to about 100 mg of DNA. The skilled clinician may also determine the suitable dosage based upon expression levels geared to meet particular plasma concentration levels of insulin or GLP-1. Normal fasting plasma concentration levels are approximately 15 pM Accordingly, the dosage of nucleic acid encoding modified GLP-1 and/or insulin to be used in the present invention may be tailored in order to achieve GLP-1 expression of about 200-500 ug per day or 5-12.5 ug/day for a DPPIV resistant analog. As shown in the exemplification, GLP-1 expression can be controlled using known techniques, such as the Valentis GeneSwitch 4.0 expression vector (e.g., see FIG. 17). In addition, methods for measuring the plasma concentration levels of insulin or GLP-1 are known in the art, and can be used to monitor and/or tailor the dosage regimen appropriately.

The vector encoding modified insulin or precursor GLP-1 can be administered using a variety of routes of administration. For example, the modified insulin or GLP-1 can be administered intravenously, parenterally, intramuscularly, subcutaneously, orally, nasally, by inhalation, by implant, by injection and/or by suppository. The composition can be administered in a single dose or in more that one dose over a period of time to confer the desired effect.

By means of the above embodiments, GLP-1 is thus expressed in the same cell in vivo upon introduction of the vector via intravenous, intramuscular, intraportal or other route of administration.

The present invention also provides compositions (e.g., pharmaceutical compositions) comprising the vectors encoding the modified insulin or precursor GLP-1 described herein. In one embodiment, the insulin or precursor GLP-1 comprises an amino acid sequence which codes for a signal for precursor cleavage by furin at the activation cleavage site of the modified insulin or GLP-1. The compositions described herein can also include a pharmaceutically acceptable carrier. The terms “pharmaceutically acceptable carrier” or “carrier” refer to any generally acceptable excipient or drug delivery device that is relatively inert and non-toxic. Exemplary carriers include calcium carbonate, sucrose, dextrose, mannose, albumin, starch, cellulose, silica gel, polyethylene glycol (PEG), dried skim milk, rice flour, magnesium strearate and the like.

Other suitable carriers (e.g., pharmaceutical carriers) include, but are not limited to sterile water, salt solutions (such as Ringer's solution), alcohols, gelatin, carbohydrates such as lactose, amylose or starch, talc, silicic acid, viscous paraffin, fatty acid esters, hydroxymethylcellulose, polyvinyl pyrolidone, etc. Such preparations can be sterilized and, if desired, mixed with auxiliary agents, e.g., lubricants, preservatives, stabilizers, wetting agents, emulsifiers, salts for influencing osmotic pressure, buffers, coloring and/or aromatic substances and the like which do not deteriously react with the DNA vector encoding modified GLP-1 and/or insulin. A carrier (e.g., a pharmaceutically acceptable carrier) is preferred, but not necessary to administer the DNA vector encoding modified GLP-1 and/or insulin. Suitable formulations and additional carriers are described in Remington's Pharmaceutical Sciences (17^{th} Ed., Mack Publ. Co., Easton, Pa.), the teachings of which are incorporated herein by reference in their entirety.

The present invention also relates to an expression vector comprising nucleic acid encoding a modified insulin or precursor GLP-1, wherein the modified insulin or precursor GLP-1 leads to generation of insulin or GLP-1a in vivo. In one embodiment, the nucleic acid sequence encodes an amino acid sequence which includes a signal for precursor cleavage by firm at the activation cleavage site of the modified insulin or precursor GLP-1.

The present invention also relates to vectors, viruses and host cells comprising nucleic acid which encodes a modified insulin or precursor GLP-1, wherein the modified insulin or precursor GLP-1 leads to generation of biologically active insulin or GLP-1 in vivo. In one embodiment, the nucleic acid sequence encodes an amino acid sequence which includes a signal for precursor cleavage by furin at the activation cleavage site of the modified insulin or GLP-1. In another embodiment, the nucleic acid construct comprises an expression construct which encodes a precursor GLP-1 and/or insulin wherein the first expression construct comprises amino acids 1-37 or other variants of human GLP-1 and a leader sequence.

Other embodiments include vectors, viruses and host cells comprising nucleic acids which encode an insulin operably linked to one or more promoters. For example, promoters that are 1) specific to a tissue or region of the body; 2) constitutive; 3) glucose responsive; and/or 4) inducible/regulatable can be used. Suitable promoters include the cytomegalovirus (CMV) promoter, the CMV enhancer linked to the ubiquitin promoter (Cubi). Tissue-specific regulatory elements for the muscle include muscle-specific promoters including mammalian muscle creatine kinase (MCK) promoter, mammalian desmin promoter, mammalian troponin I (TNNI2) promoter, or mammalian skeletal alpha-actin (ASKA) promoter. Muscle-specific enhancers useful in the present invention are selected from the group consisting of mammalian MCK enhancer, mammalian DES enhancer, and vertebrate troponin I IRE (TNI IRE, herein after referred to as FIRE) enhancer. One or more of these muscle-specific enhancer elements may be used in combination with a muscle-specific promoter of the invention to provide a tissue-specific regulatory element. Liver-specific regulatory elements may comprise strong constitutive promoters and one or more liver-specific enhancer elements. The strong constitutive promoter may be selected from the group comprising a CMV promoter, a truncated CMV promoter, a human serum albumin promoter, and an alpha-1-anti trypsin promoter. The liver-specific enhancer elements are selected from the group comprising human serum albumin [HSA] enhancers, human prothrombin [HPrT] enhancers, alpha-1 microglobulin [A1MB] enhancers, and intronic aldolase enhancers. One or more of these liver-specific enhancer elements may be used in combination with the promoter. In one embodiment, one or more HSA enhancers are used in combination with a promoter selected from the group of a CMV promoter or an HSA promoter. In another embodiment, one or more enhancer elements selected from the group consisting of human prothrombin [HPrT] and alpha-1 microglobulin [A1MB] are used in combination with the CMV promoter. In another embodiment, the enhancer elements are selected from the group consisting of human prothrombin [HPrT] and alpha-1 microglobulin [A1MB] and are used in combination with the alpha-1-anti trypsin promoter., muscle specific promoters (Souza et al., Molec. Ther., 5(5) part 2:S409 (June 2002)), liver specific promoters (WO 01/36620), and conditional promoters such as the dimerizer gene control system, based on the immunosuppressive agents FK506 and rapamycin, the ecdysone gene control system and the tetracycline gene control system. Other examples of promoters include the glucose-6-phosphatase promoter; liver type pyruvate kinase promoter; spot 14 promoter; and the acetyl-CoA carboxylase promoter. In preferred embodiments, the vectors, viruses and host cells of the invention may additionally be operably linked to one or more enhancers selected from the group consisting of an aldolase enhancer, glucose inducible response elements: Cho response elements; fatty acid synthase; prothrombin; alpha-1-microglobulin; and glucose-6-phosphatase. The vectors, constructs and viruses of the present invention may be assayed in hepatoma cells, such as H1141E cells.

Exemplification

### EXAMPLE 1

### GLP-1 Expression Constructs

Cloning of GLP-1:

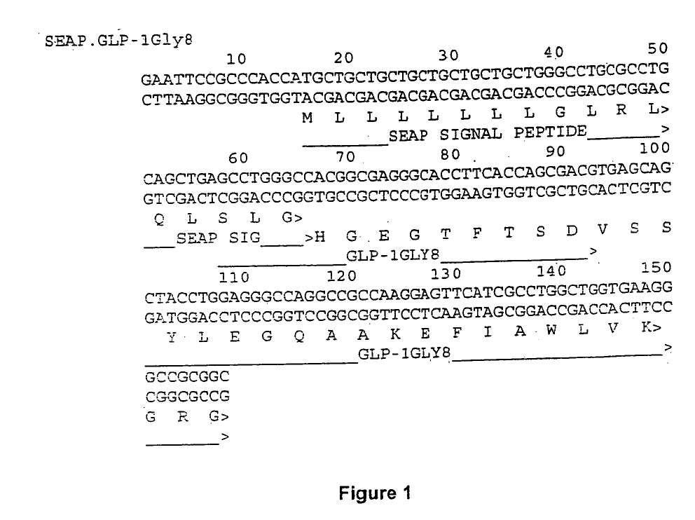

A nucleotide sequence encoding the signal peptide from secreted human alkaline phosphatase (SEAP) (Genbank Accession number CAA02290) linked to GLY-8 modified human GLP-1 (GLP-1-Gly-8) was generated by ligation of overlapping synthetic oligonucleotides. This sequence, shown in FIG. 1, is codon optimized and was cloned into the EcoRI and KpnI sites of the pCI expression vector from Promega that contains the CMV promoter, an intron and SV4O polyadenylation signal to create pCISEAPGLP-Gly-8. The signal peptide of SEAP targets the hybrid peptide for secretion and processing by signal peptidase at the SEAP/GLP-1 junction.

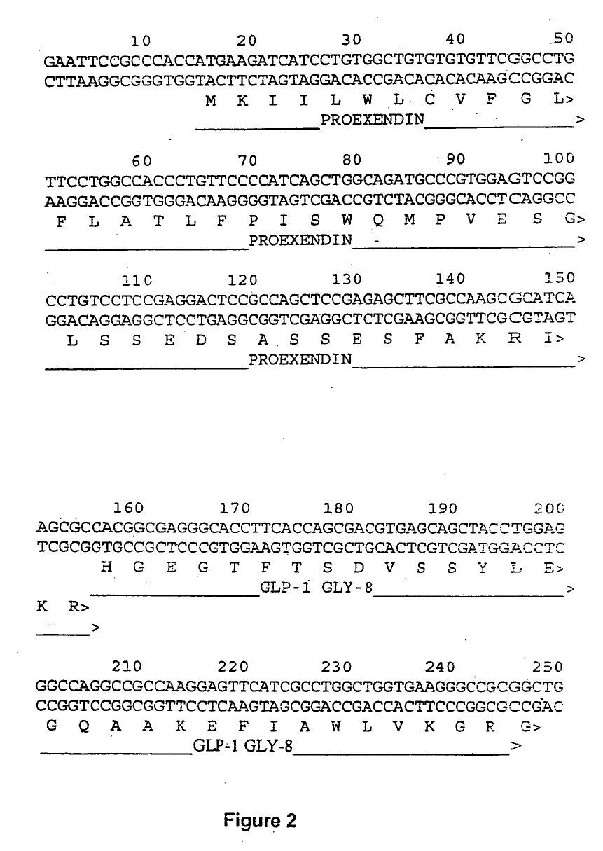

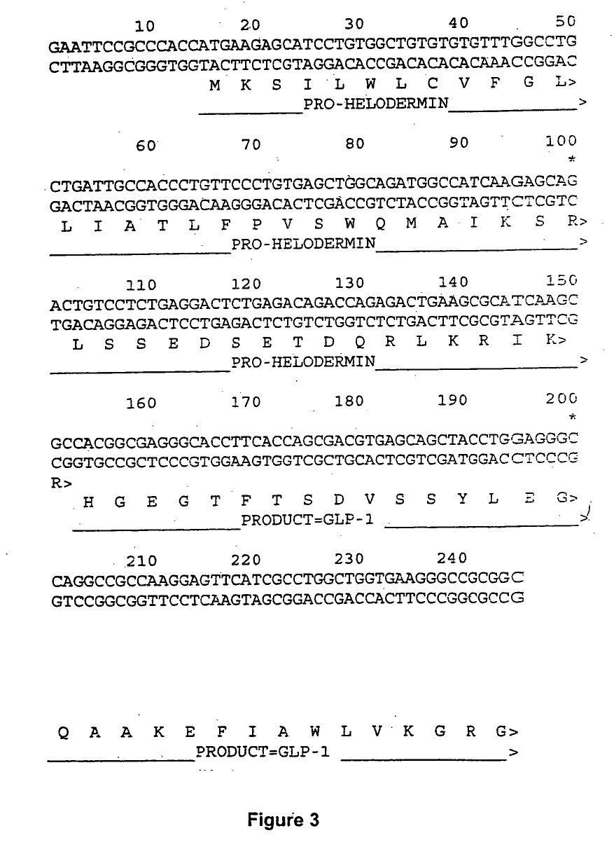

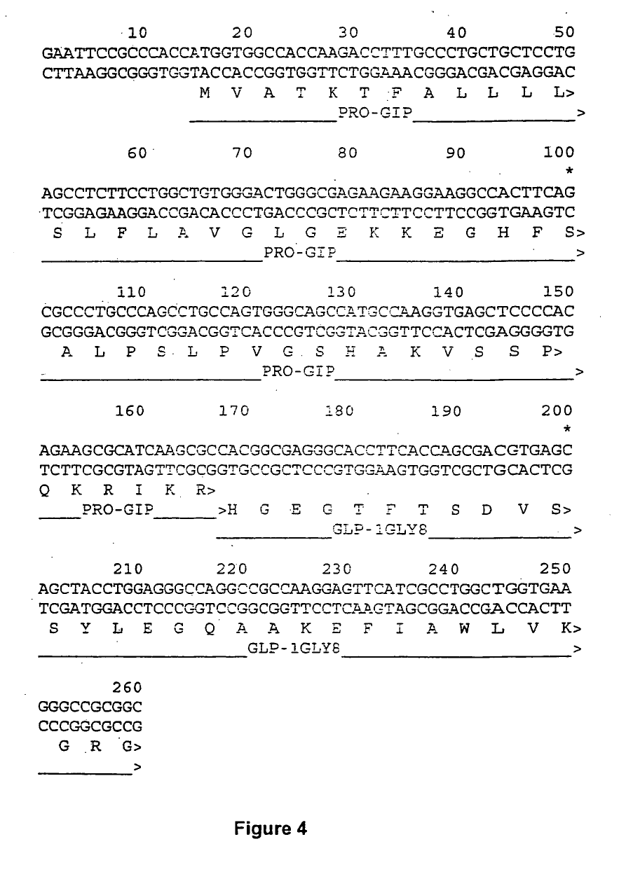

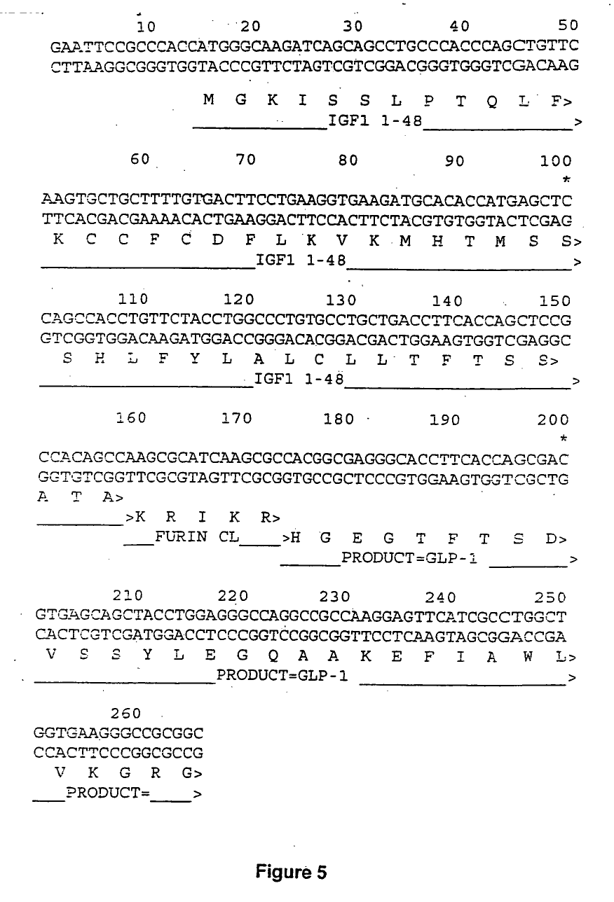

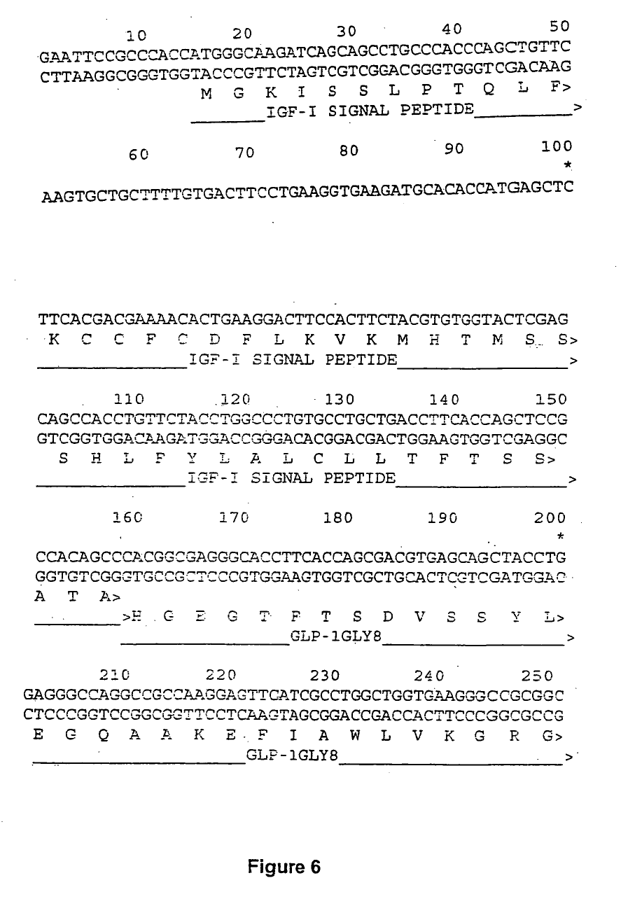

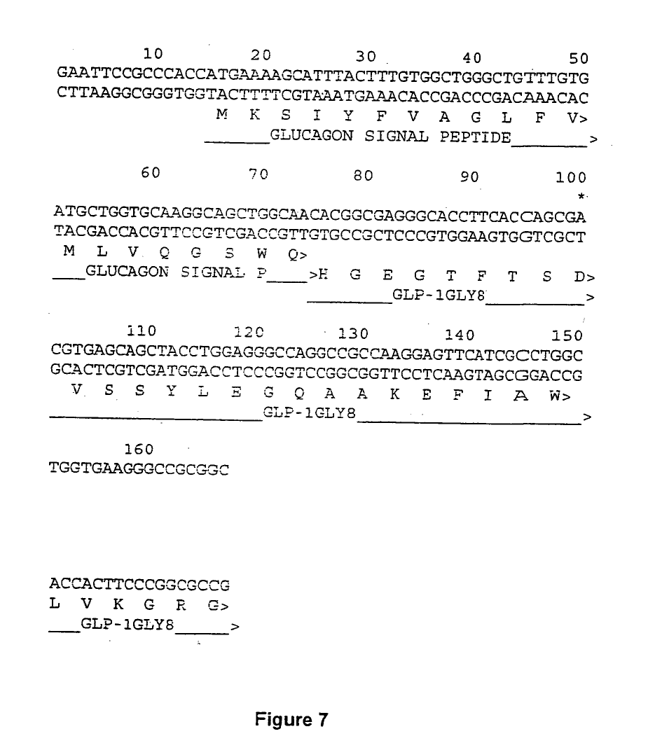

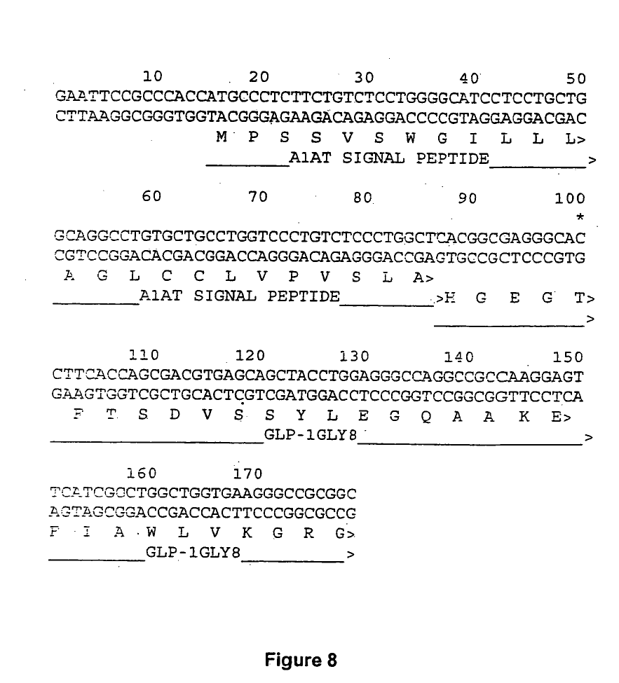

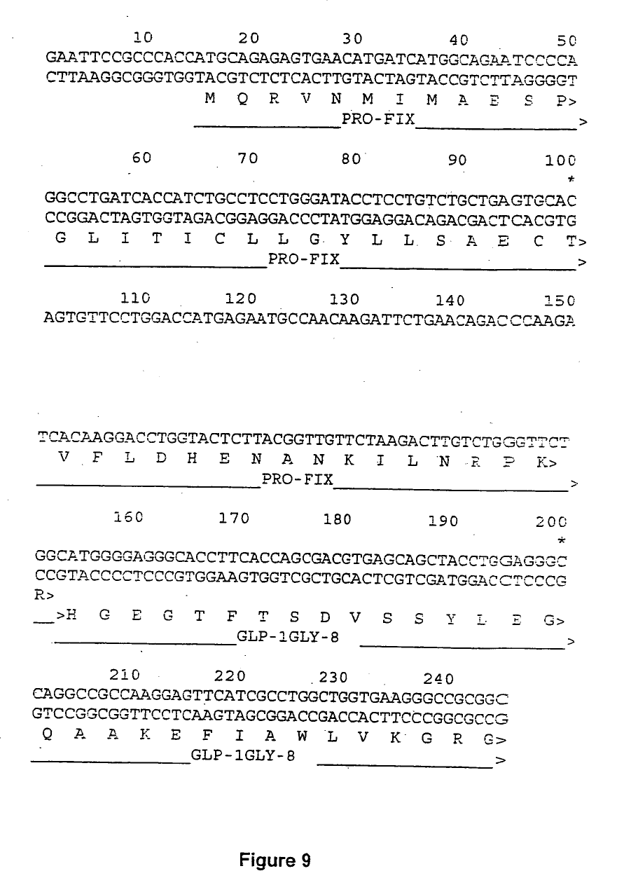

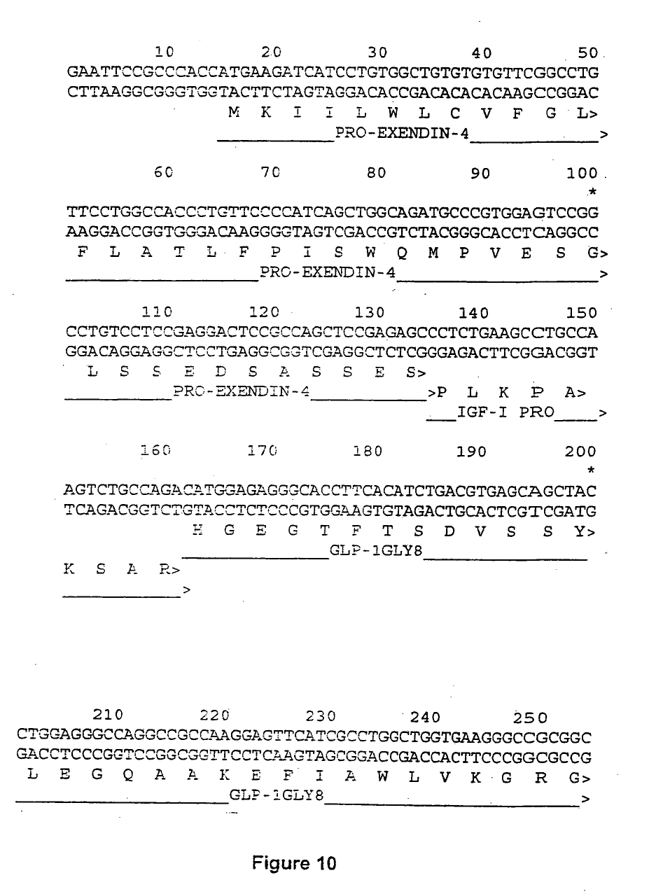

The coding sequence for GLP-1 was also linked to other leader sequences. pCISEAPGLP-Gly-8 was cut with EcoRI and BtrI which removes the SEAP leader and a portion of the GLP-1 sequence. The sequences were replaced with a fragment generated by overlapping oligonucleotides which contains the leader for proexendin-4 (amino acids 1-42) (Genbank Accession number P26349) and the missing portion of GLP-1. The sequence of the proexendin/GLP-1-Gly-8 hybrid, shown in FIG. 2 is codon optimized. In addition, the proexendin-4 sequence was modified at the GLP-1 junction to contain a consensus form cleavage site (Lys-Arg-X-Lys-Arg).

The strategy described for proexendin-4 was used to link the coding sequence of GLP-1-Gly8 to other leader sequences. Overlapping oligonucleotides containing the leader sequences for pro-helodermin (J Biol. Chem., 273(16):9778-9784 (1998)), pro-glucose-dependent insulinotropic polypeptide (GIP) (Genbank Accession number P09681), and pro-insulin-like growth factor 1 (IGF1) (Genbank Accession number IGHUI) were linked to GLP-1-Gly8 via a consensus furin cleavage site. The sequences for pro-helodermin (amino acids 1-41), pro-GIP (amino acids 1-46), and pro-IGF1 (amino acids 1-48) were codon optimized.

Overlapping oligonucleotides containing the signal peptides from preproglucagon (amino acids 1-20) (Genbank Accession number P01275), alpha-I antitrypsin (amino acids 1-24) (Genbank Accession number P01009), and insulin like growth factor I (amino acids 1-48) (Genbank Accession number IGHU1) were linked to GLP-IGly8. These signal peptides target the hybrid peptides for secretion and processing by signal peptidase at the junction with GLP-1.

Additional processing sites besides furin were also used to generate active GLP-1 from a precursor. Amino acids 1-46 of human factor IX (Genbank Accession umber P00740) contain a signal peptide as well as a cleavage site for a prohormone convertase. Overlapping oligonucleotides were used to insert this sequence into the Eco RJIBtr I digested GLP-1 vector. Insulin-like growth factor I is cleaved by a prohormone convertase at amino acid 71. This processing site (amino acids 63-71) was inserted in place of the furin cleavage site in the exendin-4/GLP-I construct.

GLP-1 Production in Cell Lines:

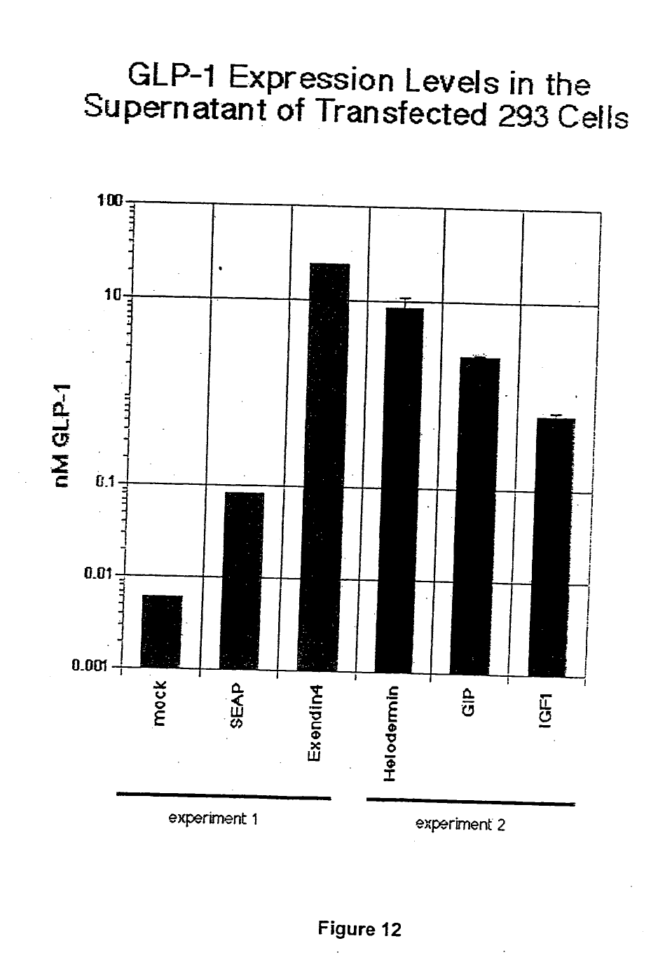

The clones mentioned above were tested using calcium phosphate mediated transfection of 293 cells, a human embryonic kidney line. On day 3 following transfection the amount of GLP-1 secreted from the cells into the culture media was quantitated using a radioimmune assay (RIA). This assay is based upon the competition between labeled 1125 GLP-1 and GLP-1 present in the culture media binding to a limited quantity of GLP-1 specific antibody. FIG. 12 shows that all constructs expressed GLP-1 at least 10-fold greater than background. The SEAP construct expressed the smallest amount of GLP-1 while the other constructs expressed GLP-1 at significantly greater levels.

Other Constructs:

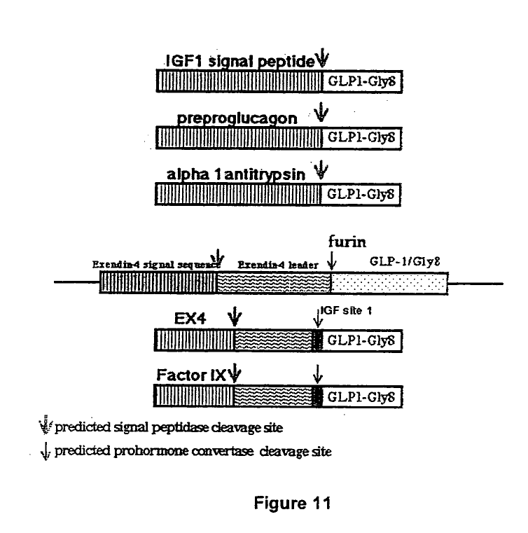

GLP-1 was also linked to the signal peptides of preproglucagon, insulin-like growth factor I (IGF-1) and alpha-1 antitrypsin. Expression vectors that require cleavage by a prohormone convertase for secretion, analogous to the exendin4 construct, were also prepared. The exendin4 construct is optimized for cleavage by furin and may not be ideal for expression in muscle. These additional constructs contain different prohormone convertase cleavage sites. One construct altered the furin cleavage site of the exendin4 vector changing it to a processing site found between the D and E domains of IGF-I. The other is a fusion between GLP-1 and the leader sequence from human Factor IX. See FIG. 11.

Expression vector containing constitutively active promoters were also made, in both plasmid and adenoviral form. These include the elongation factor I a promoter, the CMV enhancer linked to the ubiquitin promoter, and a CpG reduced form of the CMV enhancer/promoter.

GLP-1 was placed under the control of the elongation factor 1 a promoter by digesting pCIEX4GLP-1Gly8 with BglII and HindIII, removing the CMV promoter. The elongation factor la promoter was cut out from the Invitrogen vector pEF6/V5-His-TOPO using BglII and HindIII and inserted into the digested pCI vector, creating pEF1 aGLP-Gly8.

GLP-1 Production in Cell Lines:

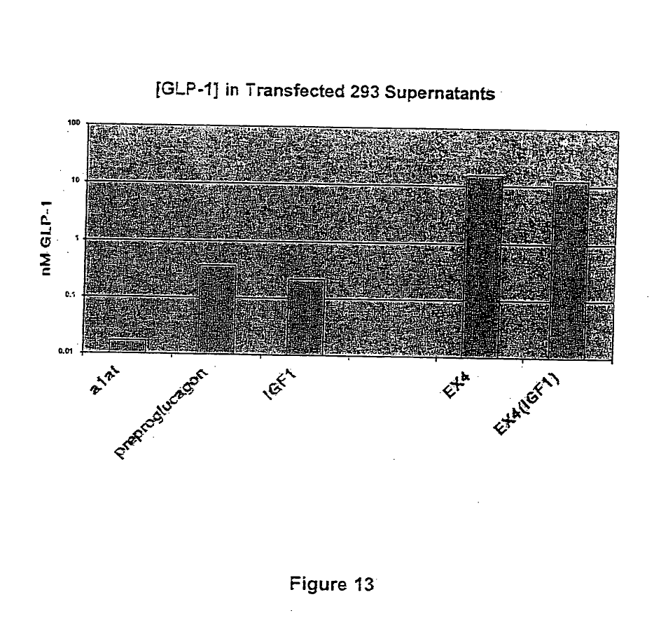

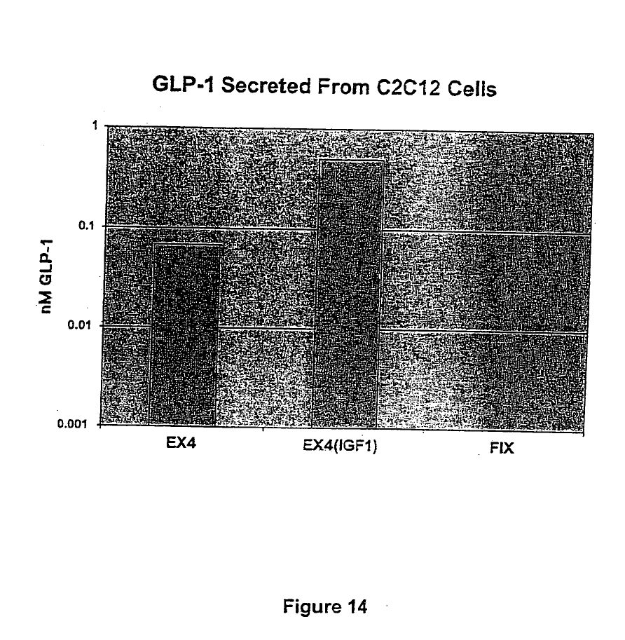

C2C12 cells are a mouse myoblast cell line and were transfected in the same manner as 293 cells. Briefly, 3×16 cells were plated on a 10 cm dish and transfected the next day using calcium phosphate precipitation. For 293 cells the cell supernatants were assayed for GLP-1 levels on the third day following transfection. For the C2C12 cells, the day following transfection the media was changed on the cells to 3% horse serum from 10% fetal calf serum. This induced the cells to fuse into elongated myotubes, similar to skeletal muscle. GLP-1 levels were assayed one week following transfection. FIG. 13 shows the concentration of GLP-1 in the culture media of transfected 293 cells, a human embryonic kidney line. The different leader sequences yield dramatically different amounts of secreted GLP-1. FIG. 14 shows the concentration of GLP-1 in the culture media of C2C12 cells, a mouse muscle line. The processing site from IGF-I yields a greater amount of secreted GLP-1 than the furin cleavage site. The Factor IX construct did not secrete detectable amounts of GLP-1 from these cells.

GLP-1 Production in vivo:

BALBc mice were transduced with 10 ug of GLP-1 expression plasmid by the method of high volume tail vein injection using the Trans IT gene delivery system from Mirus Corp. The animals were injected with the pCI series of GLP-1 vectors, which use the CMV promoter. Animals were eye-bled the following day and plasma was prepared. The amount of GLP-1 present in the plasma was determined using a radioimmunoassay (RIA) (Peninsula Laboratories, catalogue number RIK 7123).

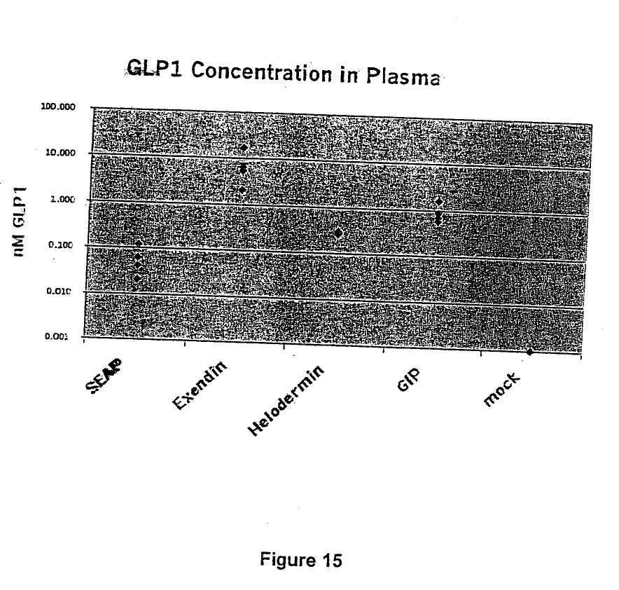

FIG. 15 shows the plasma concentrations of GLP-1 in mice transduced with GLP-1 expression plasmids by high-volume tail vein injection. High volume tail vein injections result primarily in transduction of the liver. The panel of vectors used include the vectors described herein, and transcription was driven by the CMV promoter. These samples were collected 24 hours after injection. The relative production levels parallel that observed in transfected 293 cells.

GLP-1 Mediated Correction of Blood Glucose:

The obese strain of mice db/db carries a mutation in the leptin receptor and become identifiably obese around 3 to 4 weeks of age and develop dramatically elevated blood glucose levels by 8 weeks of age. 10 week old db/db, or their lean littermates, were injected with 10 ug of GLP-1 expression plasmid or with a control secreted alkaline phosphatase (SEAP) expression plasmid. The high volume injection was similar to that described above except that the DNA was injected in 2.5 mls physiologic saline instead of using the Mirus Trans-IT system. The plasmid contained the exendin-4GLP-1Gly8 gene was under the control of a hybrid promoter composed of the human CMV enhancer linked to the ubiquitin promoter (Cubi; U.S. 20020090719 A1; U.S. 2001952152) as well as an intron from the ubiquitin gene. The exendin-4GLP-1 gene was excised as a Sca-Not I fragment and cloned into a Not I site of the CUbi (U.S. 20020090719A1; U.S. 2001952152) that had been blunted on one side. The blood glucose levels of the mice were monitored for the week prior to injection using a hand-held glucometer. Blood glucose was monitored periodically following injection. GLP-1 levels were monitored by eye-bleed followed by RIA on days 2 and 14 following injection; levels were steady in the 4-10 nM range.

FIG. 16 shows the initial test for the efficacy of GLP-1 expression vectors in treating type 2 diabetes. The db/db mouse is an obese strain of mice that has dramatically elevated blood glucose levels and is a commonly used model for type 2 diabetes. FIG. 16 shows the blood glucose levels of obese db/db mice, or their lean littermates, that were treated with a high volume injection of plasmid DNA coding for exendin4 GLP-1 under the control of the CMV enhancer/ubiquitin promoter. Control groups of mice were injected with a secreted alkaline phosphatase (SEAP) expression vector. The GLP-1 expression vector lowered blood glucose levels in both obese and lean mice with no apparent adverse effects. The glucose levels of the obese mice that received GLP-1 were lowered all the way to normal levels for a brief period and remained significantly below the SEAP injected group for several weeks.

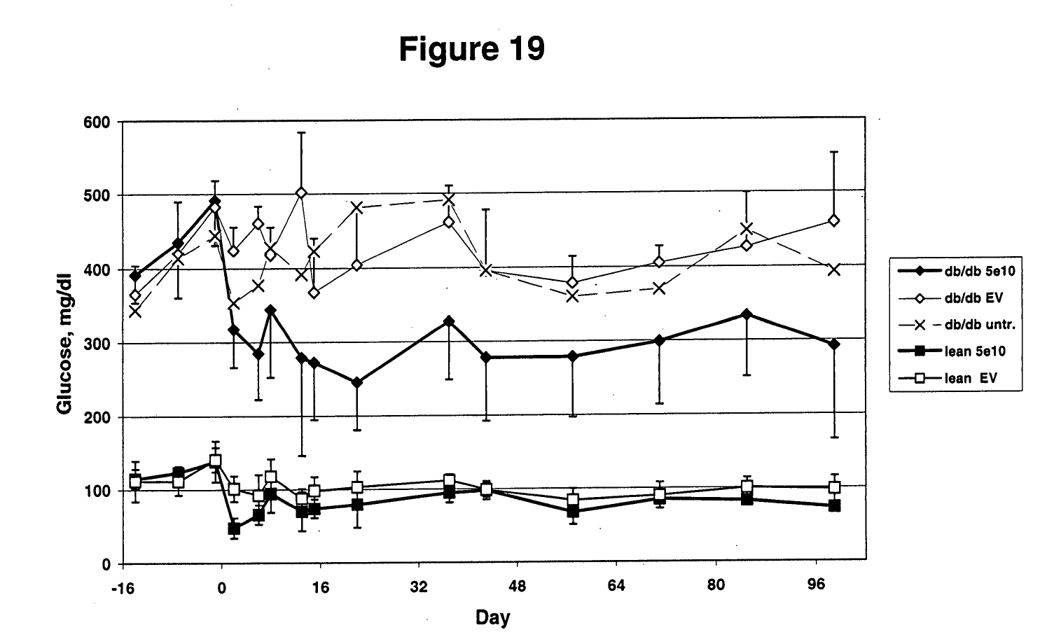

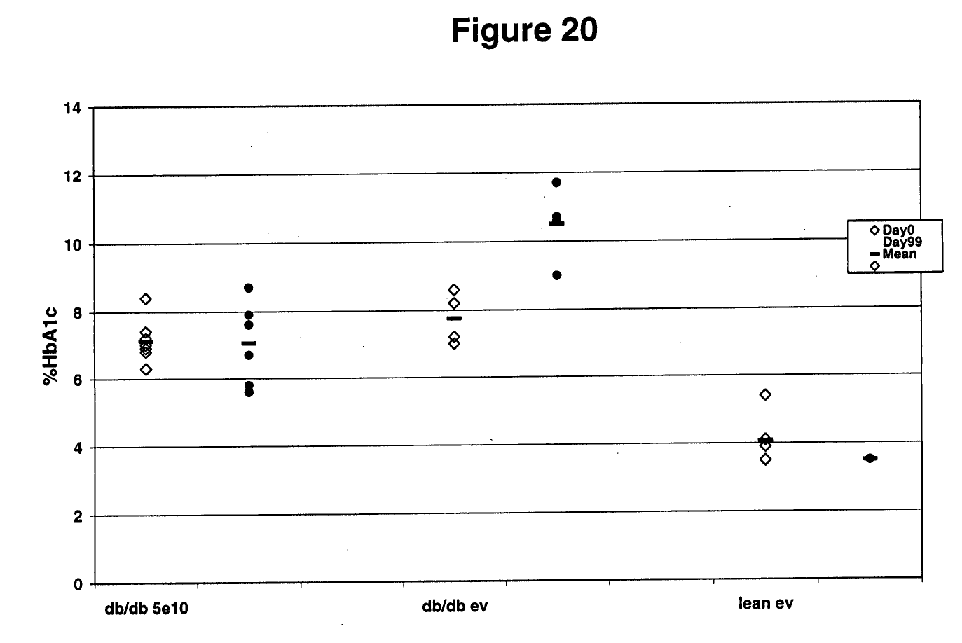

FIGS. 19 and 20 demonstrate the efficacy of GLP-1 expression vectors in treating type 2 diabetes. The db/db mouse is an obese strain of mice that has dramatically elevated blood glucose levels and is a commonly used model for type 2 diabetes. An adenovirus expression vector carrying the furin cleavable exendin4GLP-1Gly8 fusion protein (SEQ ID NO:3) under the control of the CMV enhancer ubiquitin promoter was administered to obese diabetic db/db mice, or their lean littermates as a control, via tail vein injection. Virus was administered at a dose of 5e10 viral particles. A control group was treated with adenovirus containing no transgene at the same dose. Blood glucose and glycated hemoglobin levels were measured over time in all mice out to 99 days.

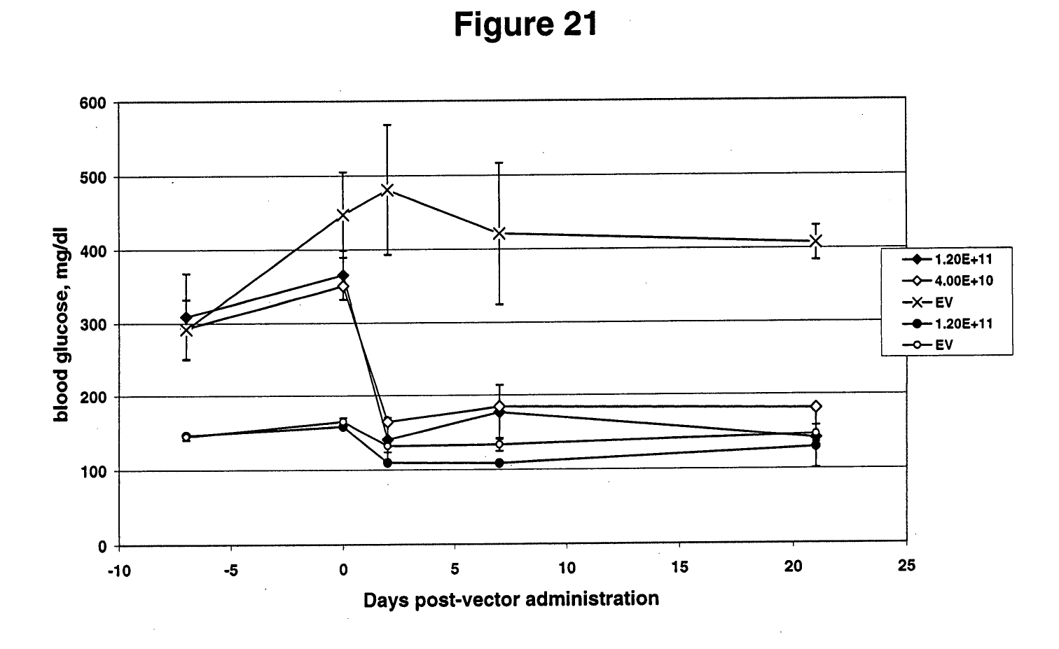

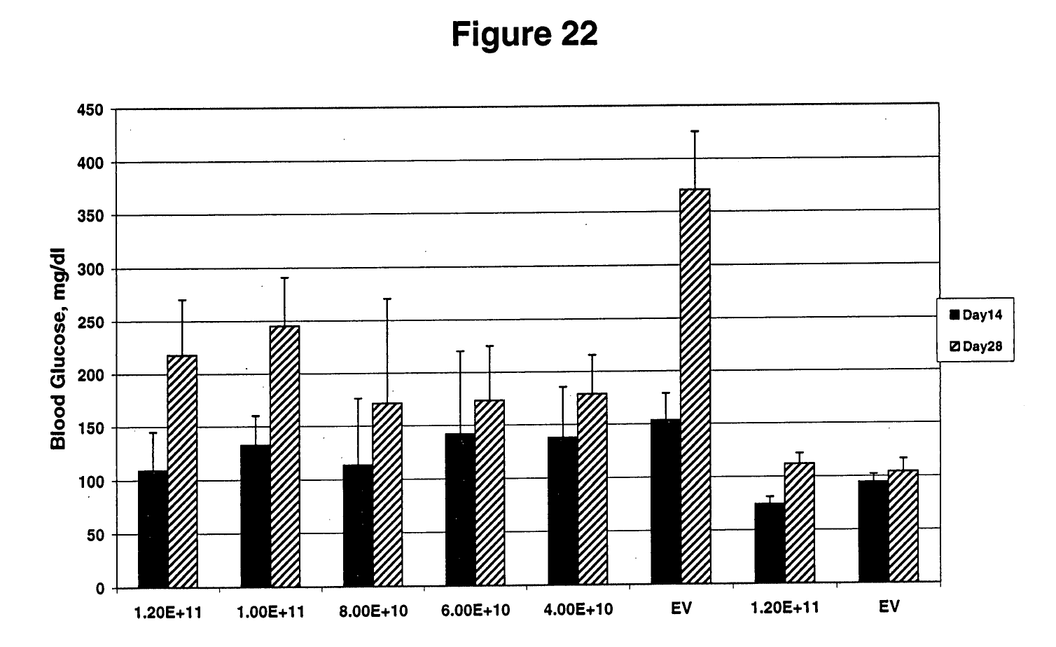

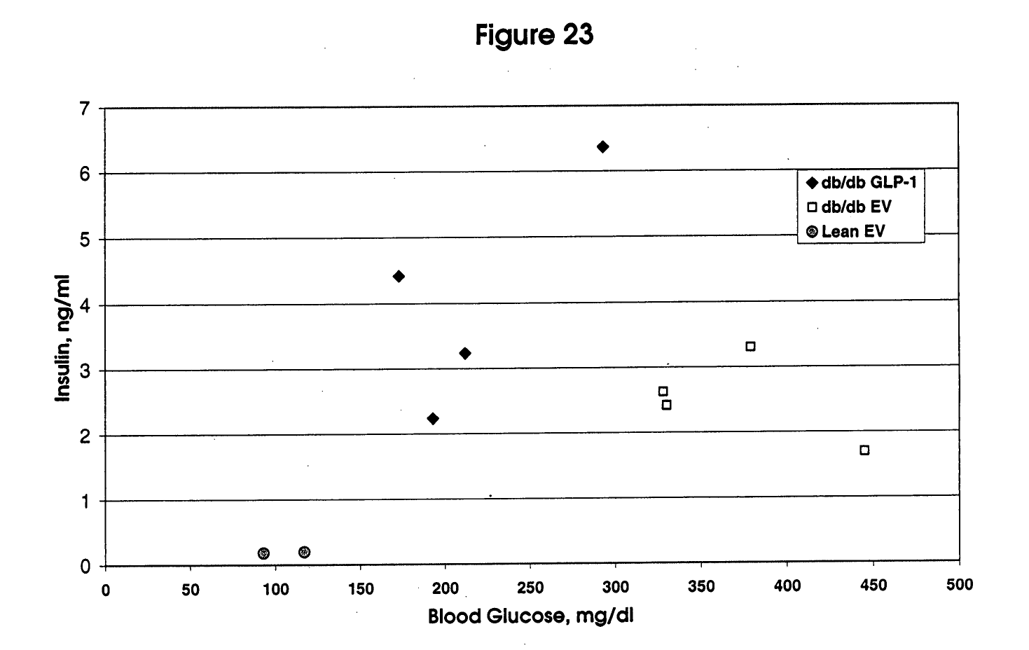

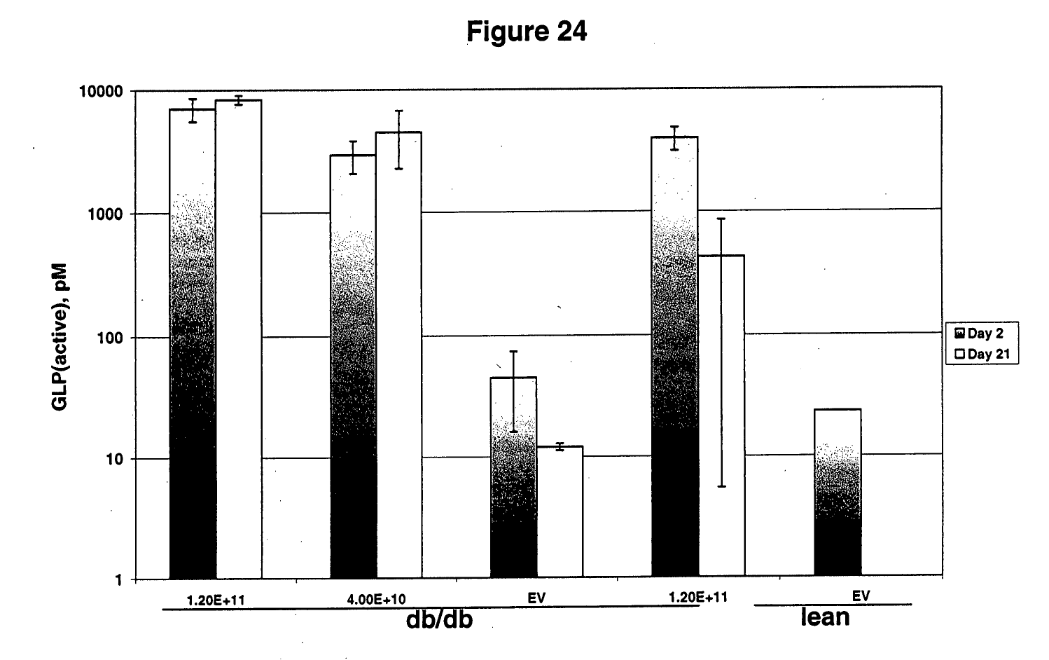

FIGS. 21-24 demonstrate the efficacy of GLP-1 expression vectors in treating type 2 diabetes. The db/db mouse is an obese strain of mice that has dramatically elevated blood glucose levels and is a commonly used model for type 2 diabetes. An adenovirus expression vector carrying the furin cleavable exendin4GLP-1Gly8 fusion protein (SEQ ID NO:3) under the control of the CMV enhancer ubiquitin promoter was administered to obese diabetic db/db mice, or their lean littermates as a control, via tail vein injection. Virus was administered at doses of 1.2e11 or 4e10 viral particles to db/db mice; it was administered at a dose of 1.2e11 viral particles to the lean littermates. A control group was treated with adenovirus containing no transgene at the 1.2e11 dose. Blood glucose levels were measured for 21 days (FIG. 21). Fasting blood glucose was measured at days 14 and 28 (FIG. 22). Insulin and blood glucose levels were evaluated at day 28 (FIG. 23). Active plasma GLP-1 levels were evaluated at days 2 and 21 (FIG. 24).

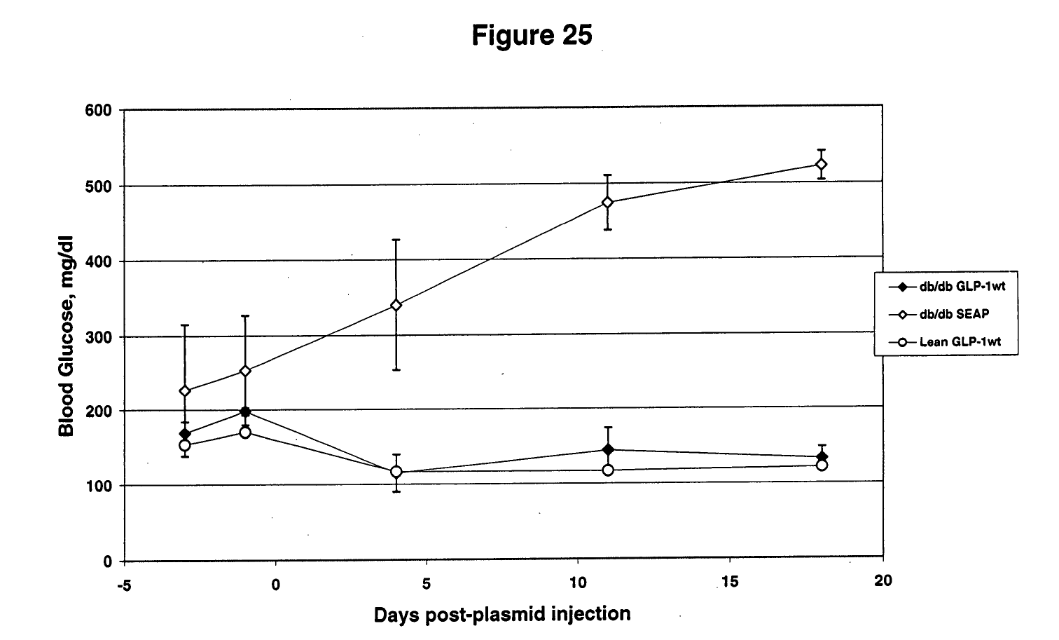

FIG. 25 demonstrates the efficacy of GLP-1 expression in blocking the progression of diabetes in a model of type 2 diabetes. The db/db mouse is an obese strain of mice that has dramatically elevated blood glucose levels and is a commonly used model for type 2 diabetes. A GLP-1 expression plasmid or a control plasmid encoding for secreted alkaline phosphatase was administered to db/db mice, or their lean littermates, using high volume tail vein injection. Plasmid was administered to db/db mice, or their lean littermates, at 4-5 weeks of age prior to the onset of beta-cell loss and elevated hyperglycemia. Mice receiving the GLP-1 expression vector did not progress to hyperglycemia but were normoglycemic with blood glucose levels similar to the lean mice which received the GLP-1 expression vector. In contrast, db/db mice treated with the control plasmid showed a rapid progression to severe hyperglycemia.

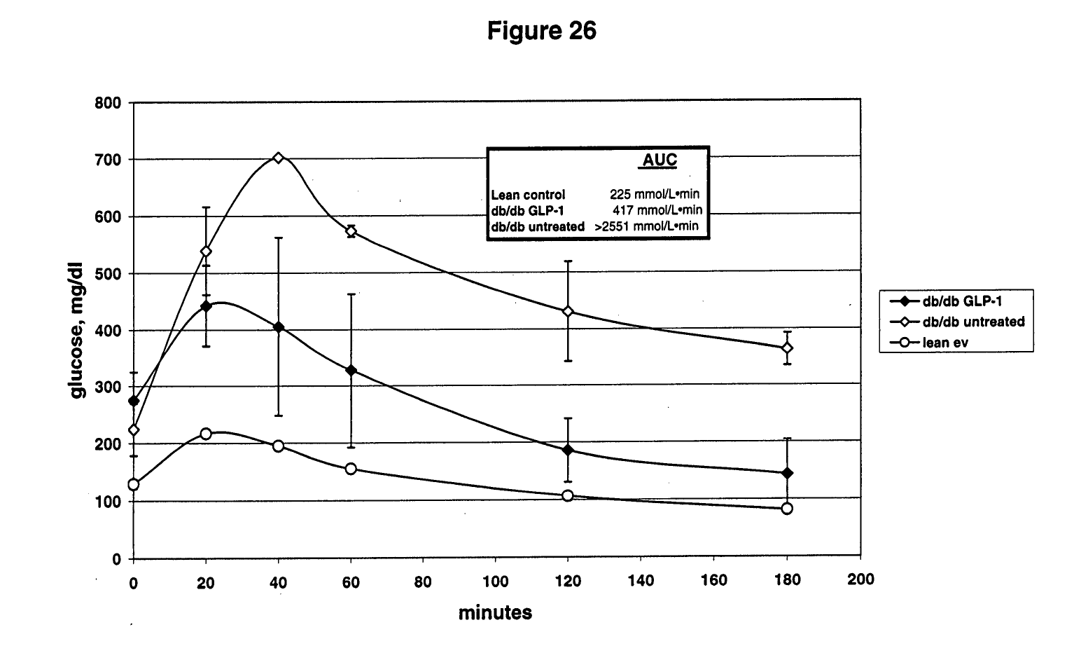

FIG. 26 demonstrates the efficacy of GLP-1 expression in improving glucose tolerance in a model of type 2 diabetes. The db/db mouse is an obese strain of mice that has dramatically elevated blood glucose levels and is a commonly used model for type 2 diabetes. An adenovirus expression vector carrying the furin cleavable exendin4GLP-1Gly8 fusion protein (SEQ ID NO:3) under the control of the CMV enhancer ubiquitin promoter was administered to obese diabetic db/db mice, or their lean littermates as a control, via tail vein injection. Virus was administered at a dose of 5e10 viral particles. A control group was treated with adenovirus containing no transgene at the same dose

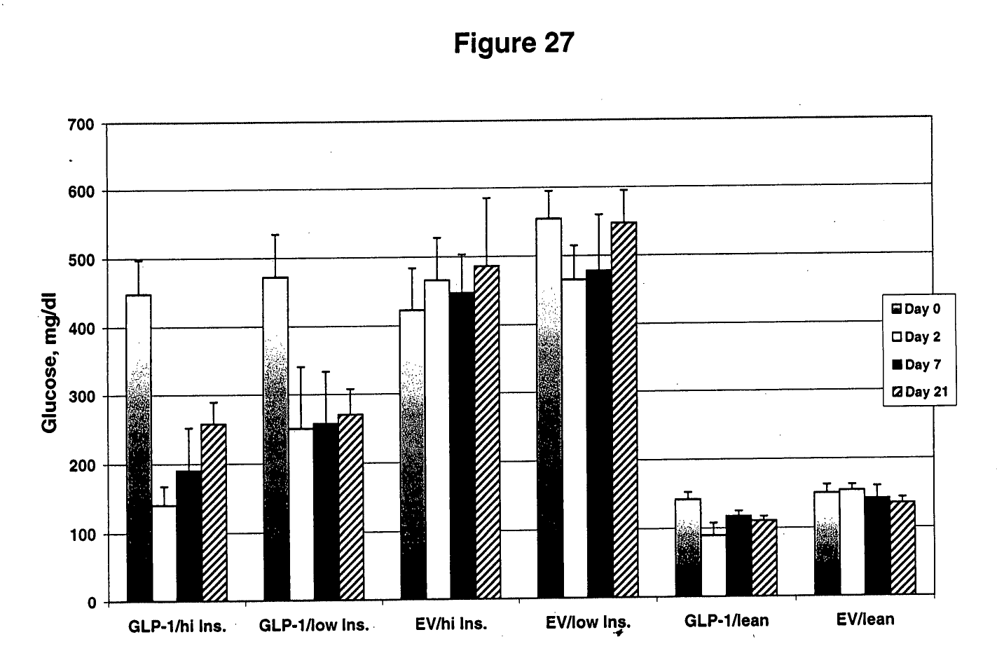

FIG. 27 demonstrates the efficacy of GLP-1 expression in lowering blood glucose in a model of type 2 diabetes in the face of differing starting levels of insulin. The db/db mouse is an obese strain of mice that has dramatically elevated blood glucose levels and is a commonly used model for type 2 diabetes. An adenovirus expression vector carrying the furin cleavable exendin4GLP-1Gly8 fusion protein (SEQ ID NO:3) under the control of the CMV enhancer ubiquitin promoter was administered to obese diabetic db/db mice, or their lean littermates as a control, via tail vein injection. Virus was administered at a dose of 5e10 viral particles. A control group was treated with adenovirus containing no transgene at the same dose

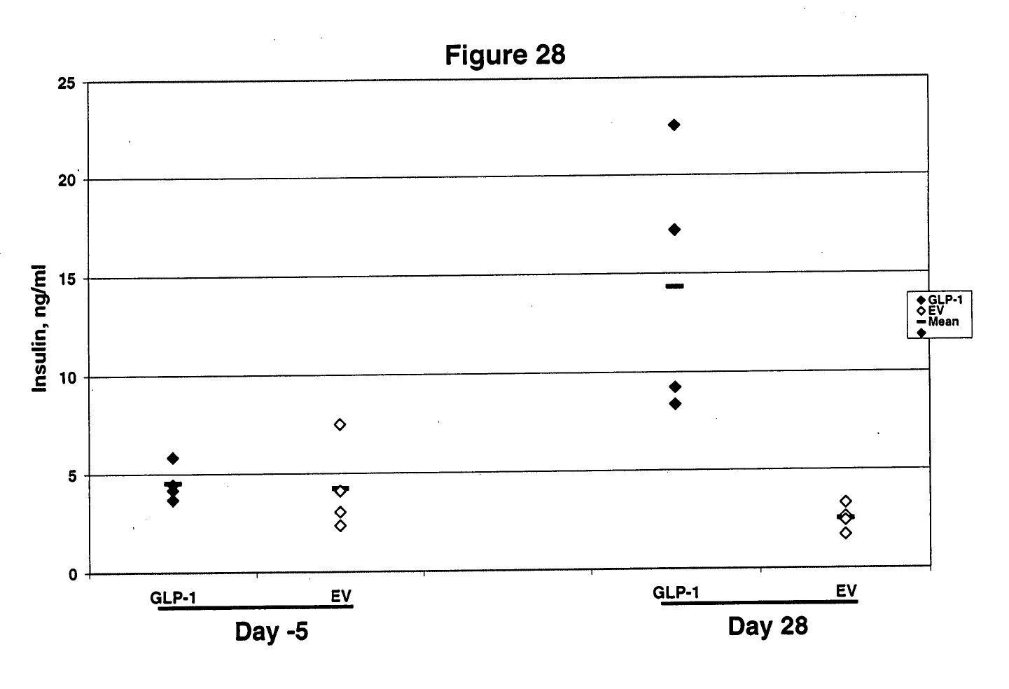

FIG. 28 demonstrates the efficacy of GLP-1 expression in improving fasting insulin levels in a model of type 2 diabetes. The db/db mouse is an obese strain of mice that has dramatically elevated blood glucose levels and is a commonly used model for type 2 diabetes. An adenovirus expression vector carrying the furin cleavable exendin4GLP-1Gly8 fusion protein (SEQ ID NO:3) under the control of the CMV enhancer ubiquitin promoter was administered to obese diabetic db/db mice, or their lean littermates as a control, via tail vein injection. Virus was administered at a dose of 5e10 viral particles. A control group was treated with adenovirus containing no transgene at the same dose.

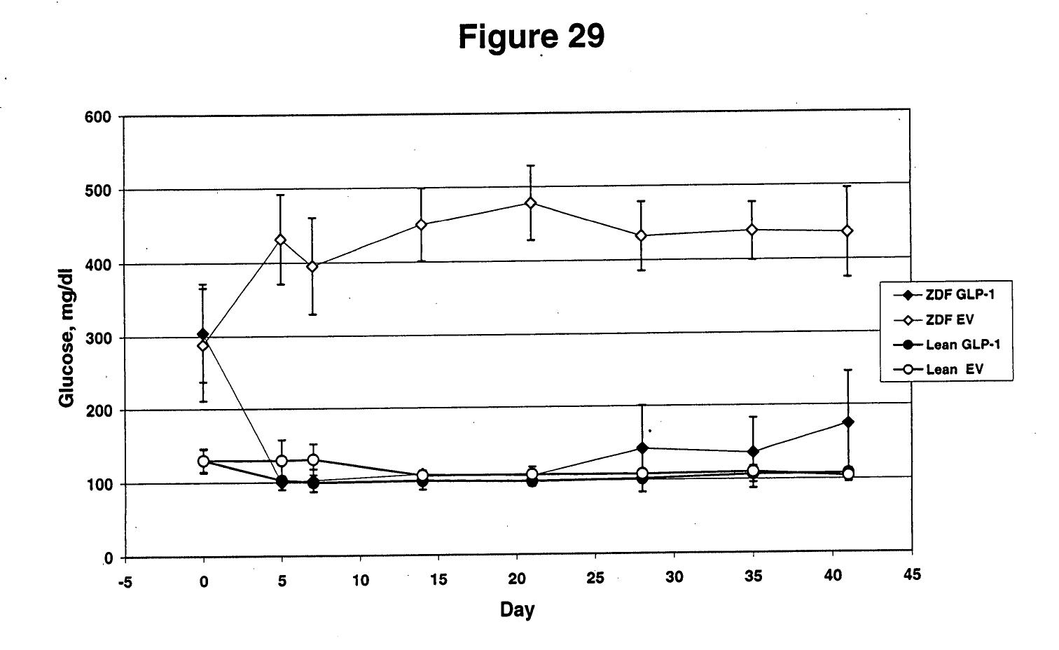

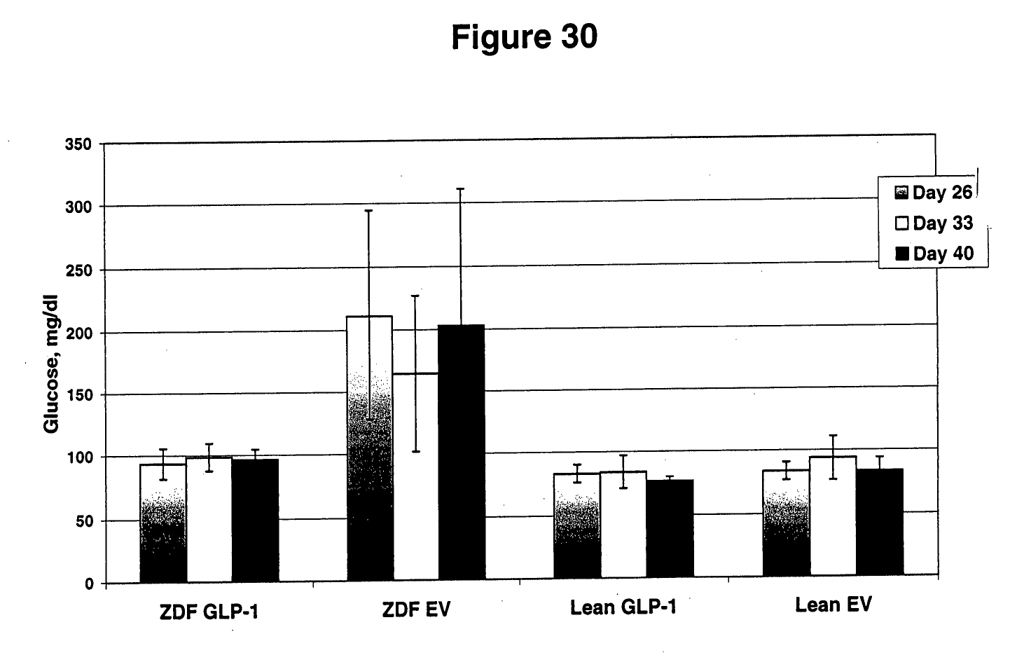

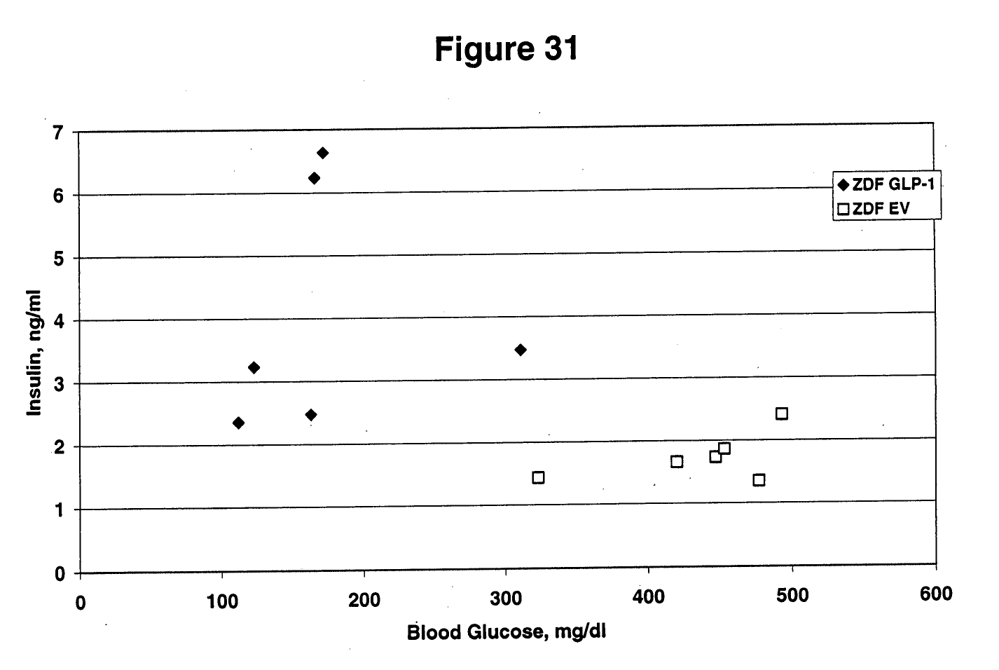

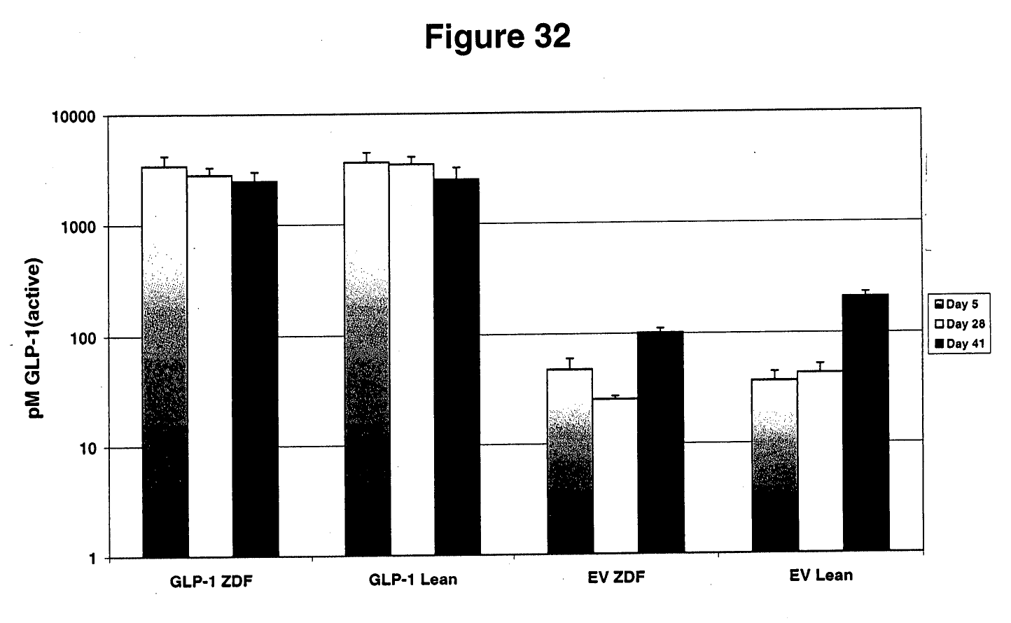

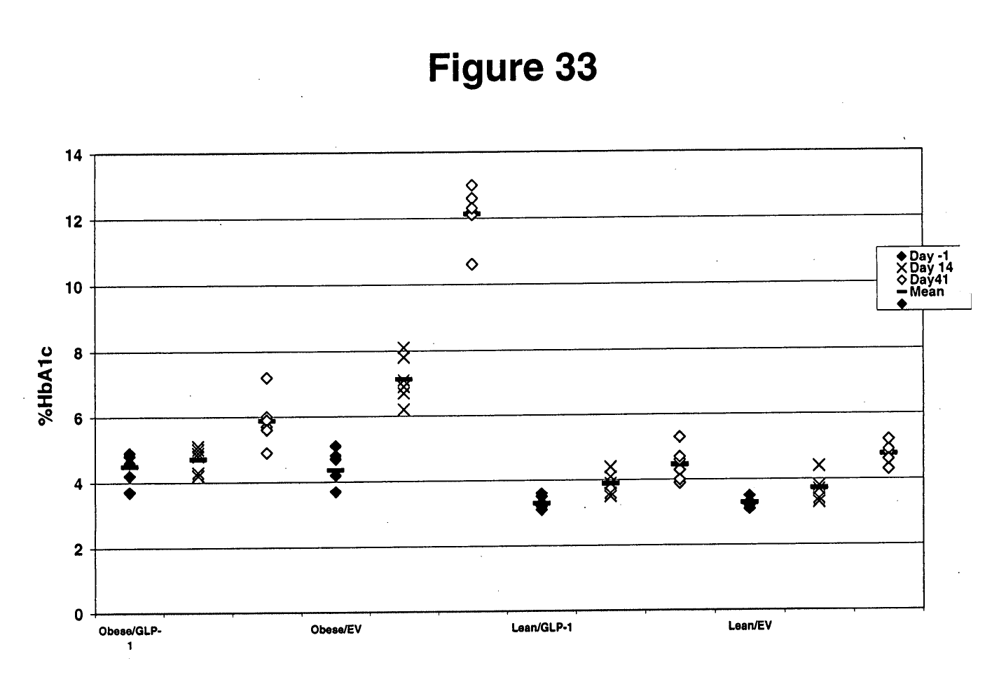

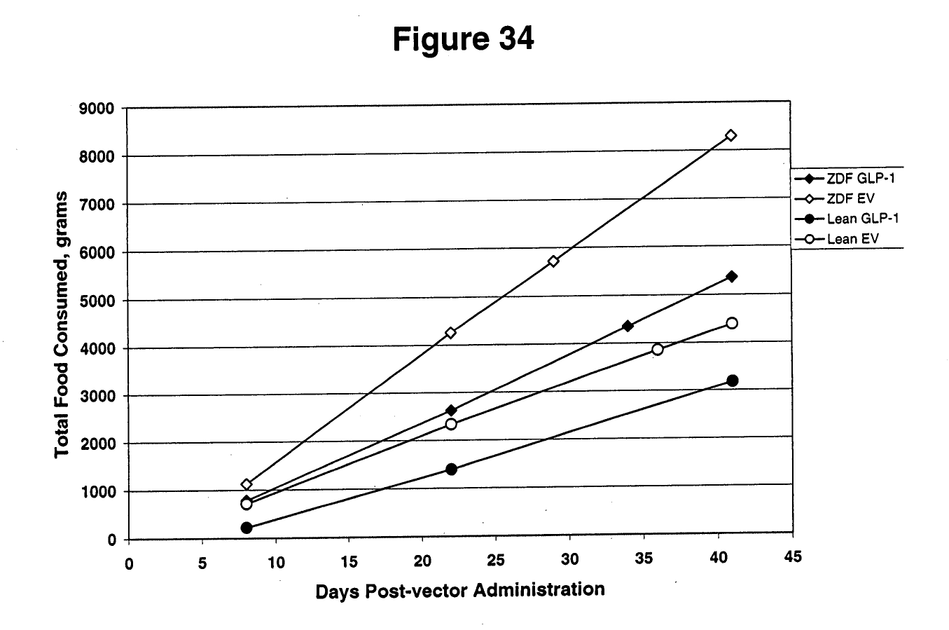

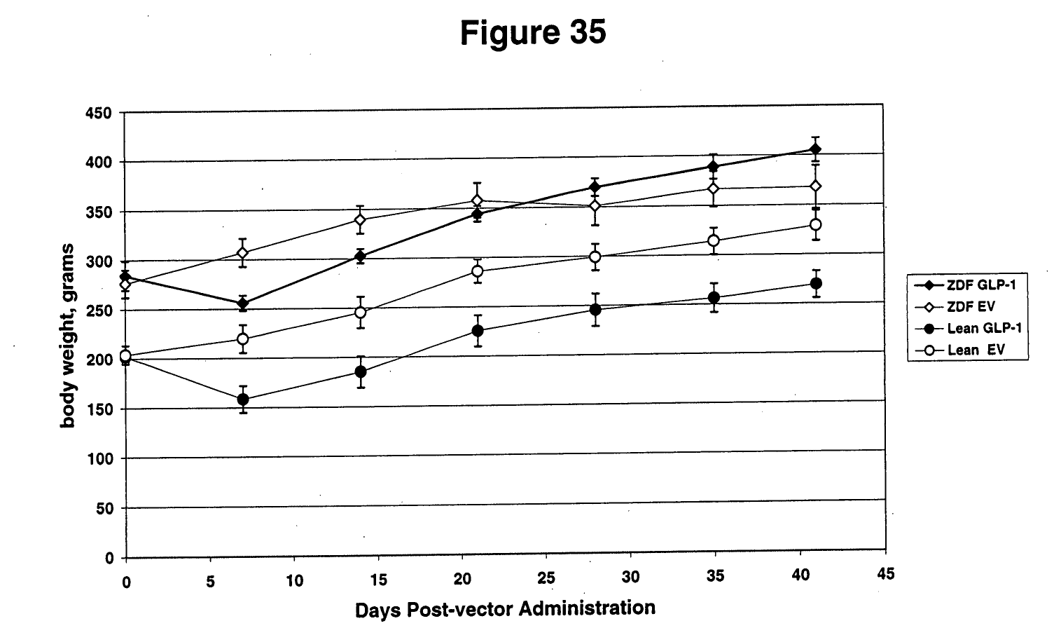

FIGS. 29-35 demonstrate the efficacy of GLP-1 expression in treating type 2 diabetes in the Zucker diabetic fatty (ZDF) model. ZDF rats have a similar progressive loss of glucose-stimulated insulin secretion similar to type 2 diabetes. Adenovirus carrying a GLP-1 expression cassette or no transgene was administered to ZDF rats at a dose of 1e12 viral particles. Over the course of six weeks after vector administration, blood glucose levels were measured (FIG. 29), fasting blood glucose was measured (FIG. 30), insulin and blood glucose levels were compared (FIG. 31), plasma levels of active GLP-1 were measured (FIG. 32), glycated hemoglobin levels were measured (FIG. 33), food consumption was measured (FIG. 34), and body weights were charted (FIG. 35).

Inducible GLP-1 Expression:

The exendin-4GLP-1Gly8 gene was inserted into the Valentis GeneSwitch vector pVC1673. This plasmid places GLP-1 expression under the control of a mifepristone inducible promoter. The promoter consists of 6 GAL4 binding sites linked to the E1b TATA box. This plasmid was co-transfected into 293 cells along with the Valentis GeneSwitch 4.0 expression vector. The transcriptional activator encoded by this vector comprises the yeast GALA DNA binding domain, a truncated human progesterone receptor ligand binding domain, and the transcriptional activation domain from the human NFkB subunit p65. Cells were transfected using the calcium phosphate technique. 24 hours following transfection, the cells were treated with varying amounts of mifepristone. 24 hours following mifepristone treatment the cell supernatants were assayed for GLP-1 levels by RIA.

FIG. 17 shows inducible expression of GLP.-1 using the Valentis gene switch system. The exendin4 GLP-1 construct was placed under the transcriptional control of a mifepristone inducible promoter. The GLP-1 vector and the GeneSwitch vector were cotransfected into 293 cells and then induced with increasing concentrations of mifepristone. The amount of GLP-1 in the supernatant was measured 24 hours after the addition of hormone.

### EXAMPLE 2

### Cloning of Insulin Expression Cassettes

Rat preproinsulin I cDNA was amplified by PCR from Sprague-Dawley rat pancreatic cDNA (Clontech) and was cloned as an EcoRI fragment into pSP7O (Promega) to generate pSP70.rppins. The C/A junction of rat preproinsulin already contains a site which is cleaved by furin therefore no further modification of this site was done. The B/C junction was modified by removing the junction from pSP70.rppins with BsmFI and PpuMI and replacing the sequence with annealed synthetic oligonucleotides encoding a junction containing a furin cleavage site (Oligonucleotide seq: 5705DA 5′ TTCTACACACCCCGCTCCAAGCGTGAAGTGGAG-3′ (SEQ ID NO: 51); 5706DA 5′-GTCCTCCACTTCACGCTTGGAGCGGGGTGT-3′ (SEQ ID NO: 52).

The pCI vector (Promega) was cut with NheI and BgIII which removes the CMV promoter and the intron. These sequences were replaced with annealed oligonucleotides containing a polylinker for cloning (oligonucleotide seq: 5′-GATCTCCTAGGGGTTTCGAAACCACTAGTAAGCTTACCGCATGCCTTAAGG-3′ (SEQ ID NO: 53) 25 and 5′-CTAGCCTTAAGGCATGCGGTAAGCTTACTAGTGGTTTCGAACCCCTAQGA 3′) (SEQ ID NO: 54). The resulting vector pCIlinker contains a polylinker sequence followed by the SV40 polyadenylation signal. The modified rat preproinsulin cDNA was cloned into pCIlinker to create pCI-rppins.

The glucose and insulin responsive rat glucose-6 phosphatase promoter (−1309 to +68 ) was PCR amplified as a HindIII-SphI fragment which was subcloned into the HindIII-SphI sites in pCI-rppins to generate PCIG-6-Prppins. Two copies of the aldolase enhancer (+1916 to +2329) were cloned 5′ to the G-6-P promoter to generate pCIAld(2)G-6-Prppins.

The disclosure of all of the publications which are cited in this specification are hereby incorporated herein by reference for the disclosure contained therein.

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated nucleic acid which encodes a precursor glucagon-like peptide 1 (GLP-1) comprising mammalian GLP-1 linked to a heterologous signal sequence.

2. The isolated nucleic acid of claim 1 wherein the GLP-1 encoded by the nucleic acid has an amino acid sequence of SEQ ID NO: 21.

3. The isolated nucleic acid of claim 1 wherein the GLP-1 is a modified GLP-1.

4. The isolated nucleic acid of claim 3 wherein the modified GLP-1 encoded by the nucleic acid has an amino acid sequence in which alanine at position 8 is replaced with glycine (SEQ ID NO: 21).

5. The isolated nucleic acid of claim 3 wherein the modified GLP-1 encoded by the nucleic acid has an amino acid sequence selected from the group consisting of: GLP-1 (7-34) (SEQ ID NO: 23), GLP-1(7-35) (SEQ ID NO: 24), GLP-1(7-36) (SEQ ID NO: 25), Val⁸-GLP-1(7-37) (SEQ ID NO: 26), Gln⁹-GLP-1(7-37) (SEQ ID NO: 27), Thr¹⁶-Lys¹⁸-GLP-1(7-37) (SEQ ID NO: 28), and Lys¹⁸ (SEQ ID NO: 29).

6. The isolated nucleic acid of claim 1 wherein the heterologous signal sequence is a sequence selected from the group consisting of: a signal peptide sequence and a leader sequence.

7. The isolated nucleic acid of claim 6 wherein the leader sequence is derived from a protein selected from the group consisting of: a cytokine, growth factor, colony stimulating factor, a clotting factor, (PACAP)/Glucagon superfamily and serum protein.

8. The isolated nucleic acid of claim 6 wherein the heterologous signal sequence is selected from the group consisting of: a secreted human alkaline phosphatase (SEAP) signal peptide sequence, a proexendin-4 leader sequence, a pro-helodermin leader sequence, a pro-glucose dependent insulinotropic polypeptide (GIP) leader sequence, a pro-insulin growth factor 1 (IGF1) leader sequence, a preproglucagon leader sequence, an alpha-1 antitrypsin leader sequence and an insulin like growth factor 1.

9. The isolated nucleic acid of claim 1 wherein the heterologous signal sequence comprises a furin cleavage site.

10. The isolated nucleic acid of claim 9 wherein the furin cleavage site encodes a peptide selected from the group consisting of: Arg-X-Lys-Arg (SEQ ID NO: 34), Arg-X-Arg-Arg (SEQ ID NO: 35), Lys/Arg-Arg-X-Lys/Arg-Arg (SEQ ID NO: 36) and Arg-X-X-Arg (SEQ ID NO: 37).

11. The isolated nucleic acid of claim 1 wherein the heterologous signal sequence comprises a prohormone convertase (PC) cleavage site.

12. The isolated nucleic acid of claim 1, wherein the nucleic acid is selected from the group consisting of
a) SEQ ID NO:1;
b) SEQ ID NO:3;
c) SEQ ID NO:5;
d) SEQ ID NO:7;
e) SEQ ID NO:9;
f) SEQ ID NO:11;
g) SEQ ID NO:13;
h) SEQ ID NO:15;
i) SEQ ID NO: 17; and
j) SEQ ID NO: 19.

13. The isolated nucleic acid of claim 1, wherein the precursor GLP-1 has an amino acid sequence selected from the group consisting of:
a) SEQ ID NO:2;
b) SEQ ID NO:4;
c) SEQ ID NO: 6;
d) SEQ ID NO:8;
e) SEQ ID NO:10;
f) SEQ ID NO:12;
g) SEQ ID NO:14;
h) SEQ ID NO:16;
i) SEQ ID NO: 18; and
j) SEQ ID NO: 20.

14. An isolated polypeptide encoded by a nucleic acid of claim 12.

15. An isolated precursor glucagon-like peptide 1 (GLP-1) comprising mammalian GLP-1 linked to a heterologous signal sequence.

16. An isolated precursor glucagon-like peptide I (GLP-1) of claim 15, wherein the precursor GLP-1 has an amino acid sequence selected from the group consisting of:
a) SEQ ID NO:2;
b) SEQ ID NO:4;
c) SEQ ID NO:6;
d) SEQ ID NO:8;
e) SEQ ID NO:10;
f) SEQ ID NO:12;
g) SEQ ID NO:14;
h) SEQ ID NO:16;
i) SEQ ID NO:18; and
j) SEQ ID NO: 20.

17. An expression vector comprising a nucleic acid of claim 1.

18. An isolated host cell comprising a nucleic acid of claim 1.

19. A method of promoting insulin production in an individual in need thereof, comprising administering to the individual an effective amount of a nucleic acid encoding a precursor glucagon-like peptide 1 (GLP-1) comprising mammalian GLP-I linked to a heterologous signal sequence, wherein the precursor GLP-1 is cleaved in vivo or ex vivo which results in generation of activated GLP-1 in the individual.

20. The method of claim 19 wherein the individual has a blood sugar defect selected from the group consisting of: Type I diabetes and Type II diabetes.

21. The method of claim 20 wherein the nucleic acid encoding the precursor GLP-1 is administered in a viral vector.

22. The method of claim 20 wherein the nucleic acid encoding the precursor GLP-1 is administered as naked DNA.
